(19) United States
(12) Patent Application Publication
(10) Pub. No.: US 2012/0109179 A1
(43) Pub. Date: May 3, 2012

(54) Intravasculature Devices and Balloons for Use Therewith

(75) Inventors: Bruce Murphy, Galway (IE); John Vard, Co. Dublin (IE); Liam Breen, Co. Limerick (IE)

(73) Assignees: TRINITY COLLEGE, DUBLIN UNIVERSITY, DUBLIN (IE); NATIONAL UNIVERSITY OF IRELAND, GALWAY, Galway (IE)

(21) Appl. No.: 13/264,528

(22) PCT Filed: Apr. 15, 2010
(86) PCT No.: PCT/EP10/54989

§ 371 Date: Jan. 3, 2012

(30) Foreign Application Priority Data
Apr. 15, 2009 (EP) 09157975.5

(51) Int. Cl. A61M 29/00; A61F 2/01

(52) U.S. Cl. 606/194; 606/200

(57) ABSTRACT

An inflatable device (1) for use within the vasculature of a body and having an expandable annular body (Ia) which is expandable by inflation of a series of inflation lumen (2) defined therein, the device having inflated (b) and non-inflated (a) states and inner (3) and outer (4) annular walls, the device being adapted so that inflationary pressure within the lumen moves the device from the non-inflated state toward the inflated state by radially outward expansion of both the outer and inner walls so that the annular body expands to form an annular structure with a central lumen defined by the inner wall (3). Non-compliant or semi-compliant balloons (36) are provided in the inflation lumen (2). The device may be employed as an embolic filter or an introducer catheter.

### FIELD OF THE INVENTION

The present invention relates to intravasculature devices and balloons for use therewith.

### BACKGROUND TO THE INVENTION

Intravasculature devices including intravasculature filter devices are known.

In recent years, a debate has emerged between the use of carotid endarterectomy (CAE) or carotid angioplasty and stenting (CAS) to determine which is the most effective treatment for carotid disease. The high risk of embolisation that is associated with CAS means that the procedure is reliant on using embolic protection systems to capture the potentially harmful micro-debris that may escape downstream to cause blockages for example those resulting in a stroke etc. These systems are also routinely used during percutaneous treatment of saphenous vein graft (SVG) disease, and further applications are being found in the treatment of peripheral vascular disease (PVD) and in accompaniment for renal angioplasty procedures.

The systems currently on the market can be categorised as either distal embolic filters or distal/proximal occlusive balloons. The benefit of using a filter is that antegrade flow is maintained following deployment of the device. In contrast occlusive balloon devices function by stagnating flow in order to trap and prevent debris from travelling downstream. The problem with such devices is that some patients are intolerant to flow stagnation and in particular long durations thereof. On the other hand, the crossing profile of occlusive balloons is smaller than that achievable with current distal filter devices. For this reason, occlusive balloons are ideally suited to crossing highly occluded or delicate lesions.

Before protection can be initiated, a distal filter must first pass the occluded lesion. Routinely a delivery sheath is used and, following crossing of the lesion, the sheath is withdrawn to deploy the filter. Similarly during retrieval, a sheath (generally larger than the delivery sheath) is advanced to collapse and capture the filter, before it can be removed from the vasculature. The majority of devices employ this strategy during delivery and retrieval [for example the devices sold as FilterWire EZ® (Boston Scientific); Emboshield® (Abbott Laboratories); AngioGuard® (Cordis Group)].

A new generation of distal filter devices have started to address some of these issues by incorporating features that minimise the need for sheaths. For instance, the Rubicon® (Rubicon Medical) filter utilises an actuating wire instead of a sheath to keep the filter in its collapsed configuration during delivery, however a sheath is still required for retrieval. Another example is the FiberNet® (Lumen Biomedical), which like the Rubicon® filter only requires a sheath for retrieval. Unlike the present invention however, the FiberNet® device must be aspirated to ensure that any loosely collected debris is not lost from its fibrous mesh during collapse and retrieval.

In instances where the lesion is too difficult to cross the preferred strategy is to use a distal occlusive balloon (such as, Medtronic's GuardWire® system, or Kensey Nash's TriActiv® system) or in very extreme lesions where crossing is not possible a proximal occlusion balloon system. This choice is often due to the lower crossing profile of a distal balloon versus a filter, which is less likely to disturb the lesion during delivery.

The apposition of the filter to the vessel wall is considered to be another crucial factor in the performance of any distal embolic filter. Failure to conform to the vessel wall causes gaps to be introduced, through which debris can pass. Some of the factors that may complicate apposition include: the type of filter employed; the shape, size and tortuosity of the vessel; and/or the orientation of the guide wire within the vessel. For instance, the protection provided by traditional nitinol-framed filters is thought to be compromised in irregular shaped vessels (i.e. vessels with an elliptical shaped cross-section). In response to this, the FilterWire EZ® (Boston Scientific) and the Spider RX® (Ev3) incorporate a looped nitinol frame, which is thought to provide better apposition in tortuous elliptical shaped vessels. The FilterWire EZ® and the Spider RX® devices have been shown to perform well in regular shaped vessels, when compared against the more traditional filter types. A study by Finol et al. (Finol E A, Siewiorek G M, Scotti C M, Wholey M H, and Wholey M H. “Wall apposition assessment and performance comparison of distal protection filters.” J. Endovasc. Ther. 2008; 15:177-185) observed that the FilterWire EZ® had the best overall average filtration rate, despite the RX Accunet® (Abbott Laboratories) showing the best overall wall apposition, yielding gaps of 0.075% of the vessel cross-sectional area. A similar study by Siewiorek et al. (Siewiorek G M, Wholey M H, and Finol E A. “In vitro performance assessment of distal protection devices for carotid artery stenting: effect of physiological anatomy on vascular resistance.” J. Endovasc. Ther. 2007; 14:712-724) indicated that the Spider RX® performed best on account of superior wall appostion when compared against four other devices (FilterWire EZ®, RX Accunet®, AngioGuard XP®, and Emboshield®). Both studies refer to the fact that none of the devices used provided complete embolic protection (despite being tested in regular circular shaped vessel cross-sections) and that vessel wall apposition should be considered the primary design variable when guarding against distal embolisation.

The disclosed present invention, which involves the use of balloon technology, exceeds performance of the marketed distal/proximal occlusive protection systems, which use elastic balloons to occlude the vessel and stagnate antegrade blood flow. In principle, the use of balloon technology to anchor a device may be viewed as less harmful to the tissue of the vessel wall, due to greater surface area conformity and a more even loading of the vessel circumference, when compared against nitinol-framed devices, which have relatively few contact points with the vessel wall. The disclosed invention has the potential to operate in a host of different vessel shapes and sizes, thereby superseding the functionality of the majority of distal embolic filter devices and also providing an alternative to occlusive protection for irregular shaped, asymmetric lesions.

Vascular access can be problematic when introducing or removing large transcatheter devices or large introducer sheaths through tortuous or diseased femoral and iliac arteries. Complications such as dissection of an artery or the removal of the intima can occur. Some different strategies have been proposed to combat vascular access limitations. For example Peterson et al (see Peterson, B. G., Matsumura, J. S. Internal endoconduit: An innovative technique to address unfavorable iliac artery anatomy encountered during thoracic endovascular aortic repair. 2008 Journal of Vascular Surgery 47 (2), pp. 441-445) have placed smaller introducer sheaths(catheters) in situ and subsequently inflated angioplasty balloons inside these sheaths to increase the internal diameter of the sheath. Subsequently this will allow the passage of large devices to the treatment site. However, this does not solve the problem of removing the sheath from the patient without potentially removing the inner layer of the artery. Moreover, there is additional cost and time involved when employing this technique. Accordingly there is a requirement for improved introducer catheters, and in particular a catheter that can be used to dilate vessels.

U.S. Pat. No. 4,723,549 describes a device comprises a collapsible filter. U.S. Pat. No. 4,794,928 describes an angioplasty balloon catheter with a trap on one end to catch debris. In certain embodiments trap is expanded by fluid. U.S. Pat. No. 5,053,008 describes a multi-sheathed catheter that has an umbrella assembly that has a meshwork for collecting debris. The umbrella is opened using a tubular balloon. A Doppler sensor and ultrasound are used to monitor the materials captured by the device.

U.S. Pat. No. 5,827,324 describes a filter system deployed by inflating a balloon. U.S. Pat. No. 5,947,995 describes a device with an inflatable cuff filter carried on a catheter. The cuff is cinched to catch blood clots, which are then removable from the body. A somewhat similar device is disclosed in U.S. Pat. No. 6,676,682. U.S. Pat. No. 6,053,932 describes a filter system on a guide wire for capturing emboli from blood. It describes a mesh structure which is expandable and thus deployable by an inflatable balloon. In certain embodiments the balloon has a thin elongate shape and is constructed of a shape-memory material configured to adapt a spiral shape. In certain embodiments the mesh is deployed by inflatable struts. A device which also uses an inflatable member in a spiral form is described in US Patent Publication No. 2001/0007947. US Patent Publication No. 2007/0038241 describes a similar arrangement with an inflatable frame of helical shape-memory material. In a somewhat similar arrangement US Patent Publication No. 2006/0047300 describes a filter device for filtering blood clots which is formed by an apical head and one or more inflatable legs connected to the head. Perfusion openings in a leg for slowly weeping anticoagulant or other therapeutic agent is disclosed. In one embodiment a secondary cavity is formed within the legs. In one embodiment the filter legs have a secondary cavity.

US Patent Publication No. 2002/0161390 describes a device with multiple filters. An expandable balloon opens out arms which deploy the filters for use. US Patent Publication No. 2002/0173819 utilises a jet to create a vacuum to suck debris in a body vessel such as an artery into a filter where it is trapped. In one embodiment a filter is used in conjunction with the device for example a mesh which comprises an arrangement with spokes extending from a central hub and attached to an outer collar of material. The collar and the spokes expand upon inflation to take up a deployed configuration of the filter. US Patent Publication No. 2004/0098026 describes a vascular filter constructed from a porous foam body.

U.S. Pat. No. 6,361,545 describes a filter device that can include a toroidal balloon or an expandable balloon. Alternatively it can include an inflatable hoop/struts arrangement. U.S. Pat. No. 6,491,712 describes a device for collecting debris flowing in an artery. The device is placed downstream from a plaque removal target site. It comprises a double walled balloon connected to a nitinol tube. A filter is secured to the balloon and is designed to collect debris from the blood which is generated when plaque is removed. The device is said to totally occlude the blood vessel, when inflated, thus ensuring all blood flows through the filter. U.S. Pat. Nos. 6,506,203, 6,136,016 and US Patent Publication Nos. 2007/0173883 and 2007/0282368 describe alternative filter arrangements.

US Patent Publication No. 2006/0149314 relates to an implantable device which can be placed in situ and anchored in a target position within an artery. It has a membrane tube which acts as a filter for filtering blood. A balloon inside the membrane tube is used to expand or contract the membrane tube. U.S. Pat. No. 5,342,301 describes a thermoplastic balloon with multiple lumens therein.

French patent publication FR 2,331,995 describes a double wall sleeve made out of an elastic material such as natural rubber, where the space between the walls has a number of lumens defined therein. Each of the lumen is inflated by inserting a needle through the inner side wall into the lumen and providing inflating a pressurised fluid through the needle.

U.S. Pat. No. 5,985,307 describes a device for the local delivery of a substance into the vasculature. The device has an inflation chamber which comprises a number of interconnected inflation cells.

International Patent Publication WO 98/55047 describes an inflatable intraluminal stent in the form of a cuff and a filter that can be deployed in the inferior vena cava using the stent.

US Patent Publication No. 2007/0038292 describes a bioabsorbable stent that can be inflated. Pores of a predetermined size can be provided for the release of materials. Pre-filled pockets which hold drugs are also mentioned.

International Patent Publication WO 2009/027531 which has a common inventor with the present application discloses a minimally invasive intravascular device. US Patent Publication No. 2005/0119688 describes a filter assembly where the filter is opened out by a ring-shaped balloon.

Notwithstanding the various devices of the prior art cited above it is desirable to provide an alternative construction of a device which may be utilised to deploy a filter. In particular it is desirable to provide a device which provides good apposition against the walls of a vessel in which it is placed. Many of the devices described above will not retain a desired shape very well and thus do not fit well in the vessel in which they are placed.

### SUMMARY OF THE INVENTION

The present invention relates to an expandable annular body which is expandable by inflation of a series of inflation lumen defined therein, the device having inflated and non-inflated states and inner and outer annular walls, the device being adapted so that inflationary pressure within the lumen moves the device from the non-inflated state toward the inflated state by radially outward expansion of both the outer and inner walls so that the annular body expands to form an annular structure with a central lumen defined by the inner wall. It is desirable that the inflation lumens comprise balloons which occupy the lumens. The inflation lumen may comprise non-compliant or semi-compliant balloons (including combinations thereof).

The device of the invention is capable of expanding to open a constricted vessel without substantial restriction of blood supply. It will be appreciated that the lumen are defined in the annular body itself (and not within the central lumen). The lumen can thus be considered to be defined in the body between the inner and outer walls. The central lumen defines blood-flow pathway through the device. The device of the invention is non-occlusive. Accordingly even if it is arranged for delivery by a carrier such as a guidewire and/or catheter it will be attached to the carrier in such a way as that the catheter does not occlude the central lumen, even if the carrier runs into the central lumen. Furthermore attachment of the device to a carrier will be arranged so as not to prevent radially outward expansion of both the outer and inner walls. In conventional carrier-delivered balloons an inner wall of the balloon remains static as it is fixed to the carrier. Such a conventional arrangement is occlusive.

In relation to the present invention non-compliant balloons are considered to include those constructed to stretch by up to 10% as measured by a change in diameter. In relation to the present invention semi-compliant balloons are considered to include those constructed to stretch by up to 20% as measured by a change in diameter.

It is desirable in embodiments of the invention that the inflation lumen comprise non-compliant or semi-compliant balloons. Such balloons will impart, as they inflate the required stretching forces to ensure the annular body expands to define the central lumen. The annular body is desirably constructed of a compliant material.

The annular body may be provided by an annular sheath of elastic material with a series of lumen defined therein.

Suitably the lumen in the annular body each house a non-compliant or semi-compliant balloon. In all embodiments combinations of non-compliant and semi-compliant balloons may be employed but for simplicity it will be appreciated that use of a single material is desirable.

A plurality of non-compliant or semi-compliant balloons are provided in at least one lumen. Desirably these balloons are interconnected. A plurality of non-compliant balloons or semi-compliant may be provided in each of at least two lumen and optionally in each lumen. Such arrangements allow for more effective expansion of the annular body as the non-compliant or semi-compliant materials will effectively control the expansion of the annular body which is typically a compliant material. Inflation of the inflatable device is achieved by inflating the non-compliant or semi-compliant balloons.

In one suitable arrangement the non-compliant or semi-compliant balloons are, in the non-inflated state of the device, arranged to be substantially flat in a substantially radial direction relative to the annular body. In such an arrangement it is desirable that the non-compliant or semi-compliant balloons are arranged to expand upon inflation in a direction substantially perpendicular to a substantially radial direction relative to the annular body.

In one embodiment the device of the invention is a double-walled elastic balloon, which is expandable from a collapsed state to an expanded state where the balloon contacts the arterial wall but maintains a central lumen at all times to allow blood flow through the central lumen. An external wall of the annular body contacts and seals against a vessel wall while an internal wall of the annular body defines a lumen.

The present invention has the potential to rival the crossing profile of balloon occlusion devices and in doing so neutralise the primary advantage they possess over current distal filter devices. The disclosed invention also allows for improved apposition to the vessel wall similar to that achievable by the elastic balloons of the occlusive devices.

The device of the present invention has at least three, preferably at least four, inflation lumen. This allows the device to expand in an even fashion about the central lumen. Generally the lumen will be sized and arranged to facilitate expansion to a desired shape. In one simple construction the inflation lumen are arranged substantially axially about the central lumen. For substantially even expansion for example to an open cylindrical shape it may be desirable to use lumen of substantially equal dimensions and to space those substantially equidistantly about the annular body.

In a further embodiment, the inner wall of the device of the present invention has a series of rebates or folds defined therein. This embodiment allows for ease of expansion and in particular ameliorates any bunching of the annular body about the inner diameter of the device. The rebates or folds are also desirably substantially evenly distributed about the annular body.

One embodiment of a device of the present invention wherein the expandable annular body is an annular double-walled balloon comprising inner and outer annular balloon walls spaced apart from each other and a series of inflation lumen defined between said inner and outer walls.

In a further embodiment of the present invention, said inflation lumen are defined, at least in the inflated configuration, by partition walls joining the inner and outer walls. Thin partition walls allow for a greater degree of inflation.

The partition walls of the present invention may be constructed of a material that is more resilient to stretching than a material from which the internal and/or external wall is constructed. This can help to ensure that the device of the invention inflates to a desired shape.

It is desirable that the device of the present invention has at least five partition walls, each joining the internal wall to the external wall. This arrangement ensures there are a series of inflation lumen between the inner and outer walls.

The device according to the present invention has at least one inflation lumen that narrows in a direction radially inwardly from the outer wall toward the inner wall at least in the inflated configuration. This again allows for ease of formation of the desired annular expanded configuration with a central lumen for the passage of blood and desirably each inflation lumen has such a configuration.

The device of the present invention has at least one, and desirably each, lumen that has in cross-section, a shape which is substantially that of a drop in at least the inflated configuration. Again such a shape allows for ease of achieving an annular shape in the expanded configuration with a central lumen defined therein.

The device of the present invention may have inflation lumens that are defined by a surface having folds therein. For example one or more lumen may have folds on their inner surface.

In alternate embodiments of the device of the present invention, the inflation lumens may be a substantially polygram shape in cross-section, for example a polygram selected from the group consisting of those in the range from substantially pentagram to substantially hendecagram. Such shapes can be selected based upon the desired end shape.

The device according to present invention wherein the inflation lumen are in fluid communication with each other so that they may be inflated together. This provides a system which is easy to use and possibly also greater control over the inflation and deflation of the device during deployment and re-deployment of the device in a vessel.

In an alternate embodiment of the present invention, the inflation lumens are not in fluid communication with each other and are independently inflatable. This provides great control over the inflation and deflation of the device during deployment and re-deployment in tortuous blood vessels.

The device of the present invention may further comprise at least one non-inflation lumen defined within the body. Non-inflation lumen an also assist is reducing the tendency of material to bulk together, particularly in those areas which may experience a net compressive force for example due to expansion of inflation lumens.

The device of the present invention has a central lumen diameter of at least about 1.0 mm, for example at least about 1.5 mm, preferably at least about 2.0 mm, such as at least about 2.5 mm, for example at least about 3.0 mm, desirably at least about 3.5 mm when in the inflated state. This ensures there is sufficient blood flow past the device. Desirably the device is connected to a delivery device/carrier such as guide wire by a mechanism which does not occlude the central lumen to any substantial extent.

In one arrangement and in the non-inflated state the body is stretch-fitted over a delivery device. Stretch-fitting ensures the device is compactly held on the device and with a low profile which aids insertion/removal.

The annular body of the device of the present invention can be tethered distally to a guide wire or alternately be tethered proximally to a guide wire or can be tethered at proximal and distal ends to a guide wire.

In a further embodiment of the present invention, the device comprises a mechanism to stretch the annular body in a substantially axial direction. Again this helps with reducing the profile for delivery/removal. Desirably also the mechanism allows contortion of the device by twisting, again to help reduce its profile.

In one embodiment, the annular body of the device of the present invention is constructed of a compliant material. This ensures compliance to vessels of different shapes. The inflation lumens of the device may comprise non-compliant or semi-compliant balloons (including combinations thereof). For example non-compliant or semi-compliant balloons may occupy the inflation lumen of a compliant annular body. The use of non-compliant balloons or semi-compliant in this manner can ensure that inflation always achieves the same degree of expansion of the device.

In a still further embodiment of the present invention, an annular sheath of elastic material with a series of lumen defined therein provides the annular body. This thus provides a two-part arrangement, a sheath and balloons which occupy lumens in the sheath. This allows flexibility. For example the same sheath may be mated with non-compliant, semi-compliant or compliant balloons (including combinations thereof) as desired. It will be appreciated that a compliant, semi-compliant or non-compliant sheath may be used. A compliant sheath may be preferable because its resilience will effect bias toward the non-expanded state when inflationary pressure is removed. If there is no such resilient bias in the device, which may occur for example where a non-compliant sheath is used then returning the device to the non-expanded configuration can be achieved utilising suitable means, for example by vacuum. Optionally the lumen in the annular body each house a semi-compliant or non-compliant balloon.

The device of the invention optionally further comprises filter and/or infusion functionality.

For example a further embodiment of the device of the present invention includes a filter which extends across the internal lumen and which can act as an intraluminal embolic capture device to capture embolic material from blood passing through the internal lumen. The filter will open out and collapse with the device.

Optionally the filter is attached to a distal end of the annular body or is attached to a proximal end of the annular body.

It is desirable that the annular body extends over a substantial portion of the length of the filter. This means that the inflation and deflation of a device of the invention will ensure opening out and collapsing of the filter. In particular this may ensure the mouth of a filter is occluded in the non-inflated configuration to ensure material caught by the filter does not escape as the device of the invention is removed following deployment.

In a still further embodiment of the device of the present invention, the device comprises an infusion means for infusing a substance into a vessel. It is desirable to be in a position to release a therapeutic agent into a vessel to assist in removal of occlusions etc.

The device may be adapted to infuse the substance when a predetermined inflated pressure in the annular body is reached. Automatically then when a pressure is reached the therapeutic material may be released. For example the device may comprise one or more rupturable reservoirs which house the substance to be released and wherein the reservoirs are ruptured in use to release the substance. Rupture may occur when a certain pressure is reached.

Alternatively or additionally the device of the invention may comprise infusion tubes on the annular body. The infusion tubes may be held to the annular body by elastic cuffs. Alternatively they may be directly bonded thereto, for example utilising adhesive. The tubes will be flexible so as to not impede the movement of the device from its non-inflated to inflated configurations and vice versa.

In an alternate embodiment, the device further comprises a perforated sheath of elastic material enveloping the annular body which allows release of the material. Alternatively the device further comprises a porous sheath enveloping the circumference of the annular body which allows for infusion. Again in such arrangements release can be triggered automatically when a certain inflationary pressure is reached or fluid can be supplied from a remote supply under user control.

In such infusion embodiments any sheath is bonded to a proximal and distal circumference of the annular body.

The present invention thus may provide a multi-lumen elastic filter scaffold. This allows the entire filter scaffold to expand and contract. The use of non-compliant or semi-compliant balloons in the multi-lumen elastic filter scaffold is a further aspect of the invention. The present invention can also provide an all-elastic device filter scaffold which is deployable by inflation. A filter, for example in the form of a mesh may be attached, for example distally, to the multi-lumen scaffold. In addition a proximal elastic region that incorporates orifices that facilitate blood flow may be provided.

A device of the invention will open out and achieve vessel wall apposition. This means that debris cannot pass between the vessel wall and the device of the invention. The device of the invention can be adapted to infuse a solution into a vessel, for example a therapeutic solution. It will be appreciated that a device of the invention can be used to treat target sites other than occluded sites. For example a device of the invention can be employed to treat an aneurism such as a cerebral aneurism. Desirably in such an application the device of the invention is adapted to infuse a therapeutic material such as a clotting agent to the target site.

The device of the invention may incorporate a distal elastic filter mesh. This is particularly desirable for applications where the treatment may result in the creation of debris.

Axial tensioning and/or twist of the annular device can be employed to reduce the profile of the device for delivery and retrieval.

It will be appreciated that the device of the invention expands by net radially outward expansion. This contrasts to expandable devices of the prior art where a balloon expands by relative movement of the walls away from each other. Such structures are not suitable for creating an annular arrangement upon expansion which will have a central lumen defined therein which will allow sufficient blood flow through the lumen and thus the blood vessel. In contrast with the present invention the central annular lumen is enlarged sufficiently to allow fluid to flow therethrough. The device of the present invention thus forms an open-ended inflated annular structure. The central annular lumen is formed when the inner wall moves radially outwards by the inflation of the annular balloon. The area between the inner and outer walls of the expandable may form an inflatable cavity which may be partitioned into inflatable lumens which in turn can be independently inflated or arranged in communication so as to be inflatable together. Upon expansion the annular body opens out to form the annular structure of the present invention.

In the present invention the expandable annular body may be formed by a compliant, semi-compliant or non-compliant material such as by a compliant balloon, semi-compliant or a non-compliant balloon. If a noncompliant or semi-compliant balloon is employed, it is desirable to use it in combination with a multi-lumen elastic component.

For certain applications the diameter of the internal lumen may be relatively large. For example the lumen may have a diameter in the range from about 4 mm to about 12 mm. Such a diameter may be useful for peripheral applications. For other applications the lumen may have a diameter from about 1 mm to about 4 mm.

It will be appreciated that the expandable annular body of the present invention will be biased by expansive forces towards an expanded configuration which is desirably substantially cylindrical in shape. However, it will be appreciated that the expandable annular body will take up the shape of a vessel in which it is placed and against which it abuts in the expanded state. For example in the expanded state of the expandable annular body may take up, upon expansion, a substantially elliptical shape of a vessel in which it is placed.

Lumen defined within the annular body can be of any desired shape. For example they may be star-shaped in cross-section.

Stretch fitting of the device onto a carrier is desirable. Stretching can help to reduce the profile of the device further. For example, axial stretching can elongate the device and reduce its (height) profile. Stretch fitting may be applied up to 50% of the maximum strain of the device. Where a filter device is employed, stretch-fitting ensures that the filter device collapses upon removal of the expansionary force on the annular body. This helps to trap debris and ensure it is not lost during removal of the device.

Desirably the expandable device of the invention is constructed of elastic material. Suitable materials include an elastic polyurethane or silicone. The filter may also be constructed of the same material. For example a polyurethane mesh may be used to form a filter. Desirably the filter takes the form of a basket.

The device of the present invention can be mounted on a carrier such as a guide wire so for example that one end is free to move axially along the guide wire when pulled, while the opposite end remains fixed to the guide wire, to reduce the profile of the annular balloon. A tether may be used in which case the tether may comprise a fluid delivery tube for delivery of expansionary fluid to the annular body.

A device of the invention may be provided in the form of a dilation catheter of the type used as an introducer catheter. The dilation catheter is adapted for the delivery of medical devices, for example valves, to a treatment site. The device of the invention can be introduced into the target vessel in an uninflated condition and then expanded when in-situ. Desirably the dilation catheter comprises a splittable body which imparts sufficient rigidity to allow the dilation catheter to be pushed into place and which splits apart with expansion of the annular body. For example the splittable body may be in the form of a tube where lengths of the tube are frangible from each other. Desirably the device of the invention comprises a splittable body on the inner or outer annular wall and desirably a splittable body on each of the inner and outer annular wall. The main rigidity of the catheter can be provided by one or both of the splittable body. This allows insertion. Inflation of the device causes the splittable body to split apart. Retraction is still possible. After splitting the parts of the splittable body will remain attached to the annular body. Desirably the splittable body is formed by an extruded tube.

The present invention extends to an expandable annular device substantially as described herein and/or as illustrated in the accompanying drawings.

### BRIEF DESCRIPTION OF THE DRAWINGS

The invention will be more clearly understood from the following description of an embodiment thereof, given by way of example only, with reference to the accompanying drawings (the drawings are not to scale and are the views of the device are increased in size relative to other drawings for the purpose of illustration where considered appropriate), in which:—

FIGS. 1A and 1B illustrate a perspective and front elevational view, respectively, of one embodiment of an annular device of the present invention in an inflated configuration; FIG. 1C shows (a segment of) sequence of cross-sectional views of the device of FIG. 1 in various states of expansion starting with a non-inflated state FIG. 1C(i), to a fully expanded state in FIG. 1C(v);

FIGS. 2A, 2B, 2C, 2D, 2E and 2F provide cross-sectional views of illustrative shape variants of the inflation lumen of a multi-lumen annular device of the invention;

FIGS. 3A and 3B illustrate a perspective and a front elevational view, respectively, of an annular device of the invention comprising interconnected inflation lumens; FIG. 3C illustrates a side-sectional view of the device of FIG. 3A while FIGS. 3D-3F show respective cross-sectional views of the device of FIG. 3 along lines A-A to C-C respectively;

FIG. 4A illustrates a perspective view of an annular device of the invention comprising independently inflatable inflation lumens; FIG. 4B shows a perspective view of an annular device FIG. 4A with the inflation lumens shown in dashed outline; FIG. 4C illustrates a front elevational view of the device of FIG. 4A; FIG. 4D shows a side elevational view of the device of FIG. 4A FIGS. 4E-4G show respective cross-sectional views of the device of FIG. 4A along lines A-A to C-C respectively;

FIGS. 5A and 5B illustrate a side view of a device of the present invention mounted on a carrier and including a mechanism to reduce the device profile, the mechanism being in a non-active state in FIG. 5A and the mechanism being in an active state in FIG. 5B resulting in a reduced device profile;

FIGS. 6A, 6B and 6C show a perspective view, front elevational view, and side elevational view respectively, of a device of the invention with a filter attachment;

FIG. 7 illustrates a side elevational view of a device of the invention with a filter attachment wherein the filter attachment is housed substantially within the lumen of the device;

FIGS. 8A and 8B provide perspective and front elevational views, respectively, of a device of the invention provided with a sheath and tethered to a guidewire; FIG. 8C provides a perspective view of the device of FIG. 8A with the sheath removed while FIG. 8D provides a perspective view of the sheath;

FIGS. 9A and 9B provide perspective and front elevational views, respectively, of a device of the invention provided with a sheath and tethered to a guidewire; FIG. 9C provides a perspective view of the device of FIG. 9A with the sheath removed while FIG. 9D provides a perspective view of the sheath;

FIG. 10A is a cross-sectional view of a device of the invention adapted to infuse therapeutic materials, in position within a vessel; while FIG. 10B shows an enlarged view of Detail A of FIG. 10A; FIG. 10C shows a schematic representation of a rupturable reservoir suitable for use with the embodiment of FIGS. 10A and 10B;

FIGS. 11A and 11B illustrate cross-sectional views of a device of the invention which is adapted for infusion, in a normal and occluded vessel, respectively;

FIG. 12A illustrates a side elevational view of a device of the invention which is adapted for infusion and including a cuff comprising infusion tubing; FIG. 12B is a perspective view of the infusion cuff of FIG. 12A;

FIG. 13A illustrates a side elevational view of a device of the invention which is adapted for infusion and including a cuff comprising infusion tubing and a perforated sheath while FIG. 13B illustrates a side elevational view of a device of the invention which is adapted for infusion and including a cuff comprising infusion tubing and a sheath constructed of porous material;

FIGS. 14A and 14B illustrate cross-sectional views of a device of the present invention in a circular and oval vessel, respectively;

FIGS. 15A and 15B are photographs of a device constructed in accordance with the present invention in a circular and oval vessel, respectively;

FIGS. 16A and 16B respectively show a transverse sectional view and a longitudinal sectional view of a device of the invention in use to treat an aneurism in a vessel;

FIGS. 17A and 17B respectively show a perspective view and an end view of a plurality of interconnected non-compliant balloons (in an expanded configuration) which may be inserted into an annular body;

FIG. 18 shows a perspective view of the device of FIGS. 17A and 17B in a collapsed configuration;

FIG. 19A and FIG. 19B respectively show a perspective view and an end view of a filter attachment suitable for use with the device of FIGS. 17 and 18;

FIG. 20A and FIG. 20B respectively show a perspective view and an end view of a sheath suitable for use with the device of FIGS. 17 and 18 and the filter attachment of FIGS. 19A and 19B;

FIG. 21A and FIG. 21B show, respectively in expanded and collapsed configurations, a device of the invention formed by assembly of the annular arrangement of balloons of FIG. 17A/17B; the filter attachment of FIGS. 19A/19B and the filter basket 23 of FIGS. 20A/20B;

FIG. 22A and FIG. 22B show, respectively from a rear perspective and front view the assembled device of FIGS. 21A/21B in an expanded configuration; FIG. 22 C is an enlarged partial view of the of the rear perspective of FIG. 22A showing greater detail; FIG. 22D is a side view of the device of FIGS. 22a and 22B while FIG. 22E is a sectional view including an inset enlarged view along the lines A-A shown in FIG. 22D;

FIG. 23 is a side view, in an unexpanded configuration, of a device of the invention in the form of an introducer catheter;

FIG. 24 is an enlarged view of the tapered tip (at the proximal end) of the device of FIG. 23;

FIG. 25 is a perspective view from a distal end, in an expanded configuration, of the device of FIG. 23;

FIG. 26 is a perspective view from a proximal end, in an expanded configuration, of the device of FIG. 23;

FIG. 27 is an enlarged view of the tapered tip/proximal end of the device of FIG. 23 in an expanded configuration with the tapered tip retracted slightly; and

FIG. 28 is a cross-sectional view of the device of FIG. 23 taken along the lines B-B with a cross section in an uninflated state indicated by (A) and with a cross section in the inflated state indicated by (B).

### DETAILED DESCRIPTION OF THE DRAWINGS

Various embodiments of the present invention will now be described with reference to the accompanying Figures. All statements about features, materials or parameters can be applied to all embodiments of the invention. In the embodiments non-compliant balloons are used in the lumen but it will be appreciated that semi-compliant balloons may additionally or alternatively be used.

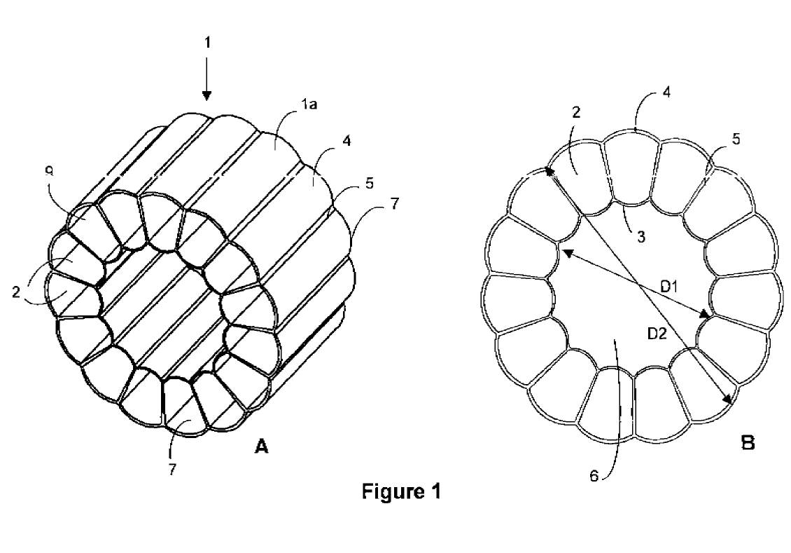

In FIG. 1 there is illustrated a perspective view and front view of a device according to the present invention. The device is for use within the vasculature of the body. The device is generally indicated by the reference numeral 1, and is shown in an inflated state. In the embodiment shown, the inflatable device 1 comprises an annular body 1a in the form of an annular balloon. The device is expandable by a series of inflation lumens 2 defined in the annular body 1a. The device has inflated and non-inflated states. Upon expansion of the expandable body the device moves from a non-inflated to an inflated (expanded) configuration. In FIG. 1 it is shown in its inflated state. As with all embodiments of the present invention the device is biased towards its non-inflated configuration so that removal of the expansion force will cause the device to move from an inflated configuration toward a non-inflated configuration.

The device 1 further comprises an inner annular wall 3 and an outer annular wall 4. Together these walls define an annular structure, which is typically in the form of a hollow cylinder. Front and rear end walls 7,8 (running transversely to the inner and outer walls 3,4) close the annular structure. An inflatable cavity 9 is formed between inner and outer walls 3,4. In the embodiment, the cavity 9 is partitioned to define inflatable lumen. In particular partition walls 5 run (radially) between the inner and outer walls and substantially axially along the device 1 so as to form longitudinal lumens 2 within the annular body 1a. In the embodiment the longitudinal lumens 2 are inflatable. A central lumen 6 allows for blood flow through the device. When in position within the vasculature the outer wall 3 abuts and forms a barrier with the vessel wall thus cutting off blood flow save through the lumen 6.

In this embodiment the inner and outer walls 3,4 are each formed by a thin membrane of material. It will be appreciated that the partition walls 5 are also constructed of a thin membrane of material. The material may be thin even in the non-inflated state, and according to one desired aspect of the invention the material thins as the device expands. In this respect the volume of the annular body has a greater cavity volume than material volume.

In certain embodiments it may be desirable that the partition walls are created from a material that is more resilient to stretching than material from which the internal and/or external wall is constructed. Selection of materials in this way can help to adjust the inflation profile of the device.

It is desirable that there are at least five partition walls each joining an internal wall to the external wall. This allows for more even distribution of the stretching forces and thus even expansion of the device.

The annular body is formed by side-by-side and circumferentially arranged lumen 2. It will be appreciated that as the device 1 expands an internal diameter D1 increases. So too does an external diameter D2.

It will be appreciated that the length of the diameter D1 or D2 can be varied depending on the degree to which the device is expanded. In all embodiments, it is desirable that the device is sufficiently expanded so that the internal lumen 6 (which will have the same diameter as D1) allows sufficient blood flow through the device.

The inflation lumens 2 narrow in a direction radially inwardly from the outer wall 4 to the inner wall 3. Each thus forms a circumferential segment of the annular device. In the inflated state, the annular body expands radially outward to grip a vessel wall and enlarges sufficiently to allow blood flow through the central lumen 6. The shape of the inflation lumens 2 as depicted in FIG. 1 is substantially that of a drop. Such a shape allows for desirably expansion of the device.

One of the issues then arises with inflating devices, for example with fluid pressure, it is to achieve the desired end configuration of the device. Some constructions of prior art devices include balloons which are mounted on a guide wire. Typically, such mounting of the balloons means there is no central lumen through which bodily fluids such as blood can pass. Often, in such cases, the balloon is mounted on a rigid carrier, such as a rigid conduit. In such cases one wall (an inner side) of the balloon may be fixed in position and it is only the other (outer side) wall that can expand in response to expansionary pressure. In such a case only the external diameter of the balloon tends to increase when inflation occurs. In other devices the balloon does not have an annular shape, instead it is provided with a neck through which inflationary fluid is provided. In such a case the opposing balloon walls expand away from each other to a substantially equal extent.

FIG. 1C shows in sequence (expansion in the sequence from (i) to (v)) the type of expansion which is achieved with a device of the present invention. For convenience of illustration only a segment of the device is shown in this sequence. FIG. 1C(i) shows the original configuration of the unexpanded device while FIGS. 1C(ii)-(v) show the original configuration of FIG. 1C(i) in ghost outline (for comparative purposes), while the expanded configuration is shown in full outline.

FIG. 1C (i) shows the annular body 1a in an unexpanded configuration. (It will be appreciated that the profile of the annular body 1a may be further reduced for delivery/travel within the vasculature as will be described in more detail below). FIG. 1C (i) shows the annular body 1a before any expansion force is applied. FIG. 1C (ii) shows the effect of expansionary forces on the device. In particular each of the lumen 2 is being inflated by an inflationary fluid. As can be clearly seen by comparison with the device as shown in FIG. 1 C(i) the inflationary pressure causes outward radial expansion of both the inner and outer walls 3,4. FIG. 1C(iii) shows the effect of continued expansion as does FIG. 1C(iv) and FIG. 1C(v). It will be appreciated that upon removal of the expansionary force the sequence will be reversed as the device contracts.

While there are a number of effects upon inflation, the desired overall effect for all embodiments of the invention and which is clearly demonstrated in the Figures is that there is substantial net expansion of the cylindrical ring of the annular body radially outwardly. FIG. 1C(v), in particular highlights the substantial outward net radial movement of the entire annular body that has occurred from the non-inflated to the inflated states. (The configuration of FIG. 1C(v) is the same as that shown in FIGS. 1A and 1B). The position of the inflation lumens is critical to this process and accordingly the arrangement of the lumens within the annular body will be selected to cause substantial outward net radial movement of the entire annular body upon inflation of the inflation lumens.

As is desirable in all embodiments the inner diameter (D1) of the device in the inflated state is substantially greater than the outer diameter (D2) of the device in the non-inflated state. Typically the outer diameter will be in the range from about 1 mm to about 12 mm, preferably about 4 mm. Typically the inner diameter will be in the range from about 0.6 mm to about 10 mm, preferably about 3 mm. Typically the device when expanded has an outer circumference of the order of from about 3 to about 10 times that when in an uninflated state. Typically, when expanded, the cross-sectional area of the device occupies no greater than about 50%, suitably no greater than about 40% of the cross-sectional area of a blood vessel in which it is placed. When moving from the uninflated to inflated states it is desirable that the degree of expansion that occurs is such that the outer circumference of the device increases at least about three-fold in size, for example, at least about four-fold.

The device of the invention may be composed of compliant or non-compliant material. Non-compliant material may be utilised to ensure that the device has set inflated dimensions. Compliant material may be used where compliance of the device may be desirable to ensure that it can expand to a variable extent by stretching so that the device of the invention may be used in different sized and/or shaped vessels. Desirably the annular body 1a of the expandable device 1 in the present embodiment is composed of a compliant material.

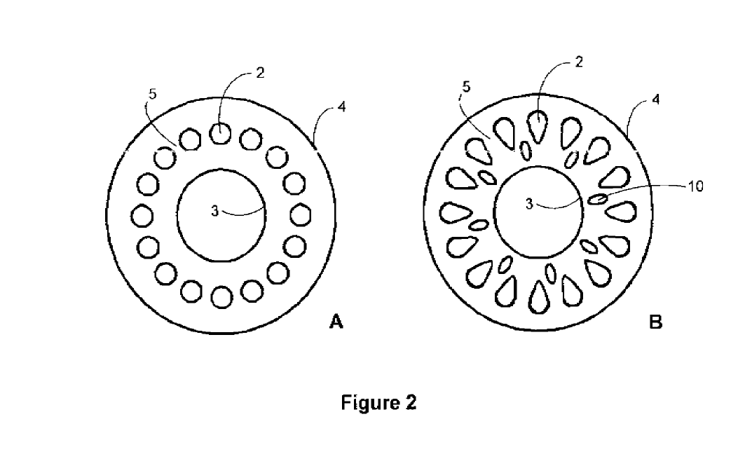

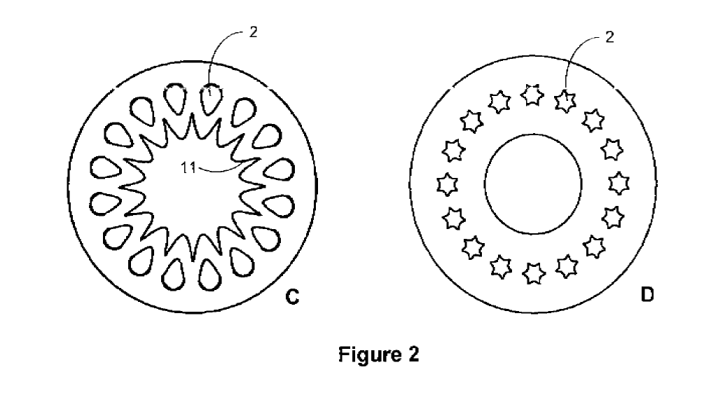

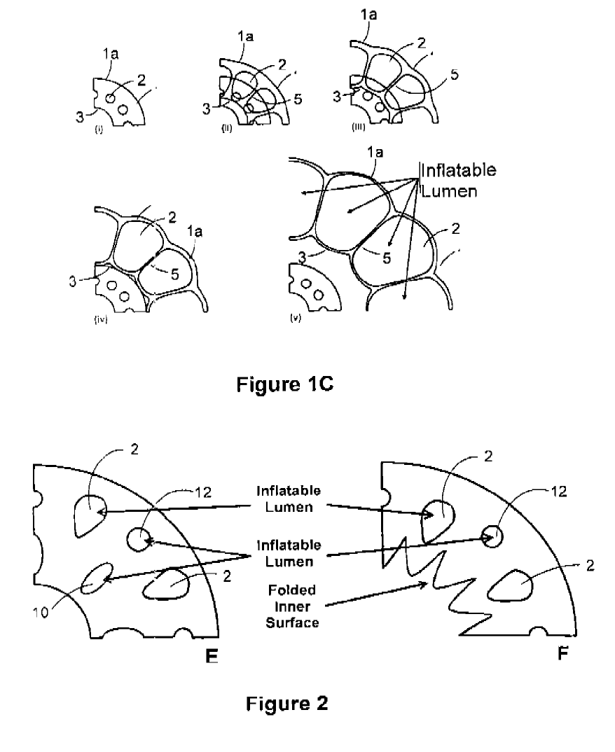

With reference to FIG. 2, where the inflation device is in a deflated state, it can be seen that a variety of shapes may be adopted for the inflation lumens 2 of the inflatable device 1. FIG. 2A, is a cross-sectional views of a device 1 which is similar to that in FIG. 1. For example, the inflation lumens 2 are depicted as being circular in shape in FIG. 2A, a teardrop shape in FIGS. 2B and 2C, and the inflation lumen have a shape having folds or rebates 11 therein such as in the substantially polygram shaped lumens, star-shaped lumens in FIG. 2D. As the portion of the annular balloon that experiences most stretch is the material lining the surface of the inflation lumens 2, the lumen shape is not a significant factor on the ultimate shape of the expanded device. The inflation pressure expands the inflation lumens 2 in a radial manner so that the expanded inflation lumen 2 tends toward a circle regardless of the shape of the inflation lumen 2. Desirably the non-inflation lumens 10 are evenly distributed around the annular body. It is also desirable that as shown in FIG. 2B, the non-inflation lumens 10 are disposed radially inwardly of the inflation lumens 2.

In a further embodiment of the present invention, the inflatable device 1 further comprises a series of non-inflation lumens 10 (FIG. 2B) and/or folds/rebates 11 (FIG. 2C) on the inner surface of annular body 1a. The non-inflation lumen 10 and folds/rebates 11 are effective to reduce the amount of material in the annular body thus allowing for better expansion. There is less material to be compressed between the inflation lumen and there is therefore less bulging upon inflation. Such arrangements also help to ensure that the inner diameter is pulled outward with the outer diameter during expansion. For example the folds or rebates 10 are effective in reducing the material about the internal diameter, which also ensures the internal diameter is pulled outward with the outer diameter during expansion.

FIG. 2E shows a further embodiment where there are a series of inflatable lumens 2 and two series of non-inflation lumens. In particular there is a first series of non-inflation lumens 10 (which correspond generally to those shown in FIG. 2B). A second series of non-inflation lumens 12 are also shown in FIG. 2E. These non-inflation lumens 12 are defined within the annular body at a position radially outwardly from the first series of non-inflation lumens 10.

FIG. 2F shows an embodiment with folds/rebates 11 similar to those of the embodiment of FIG. 2C but additionally having non-inflation lumen 12.

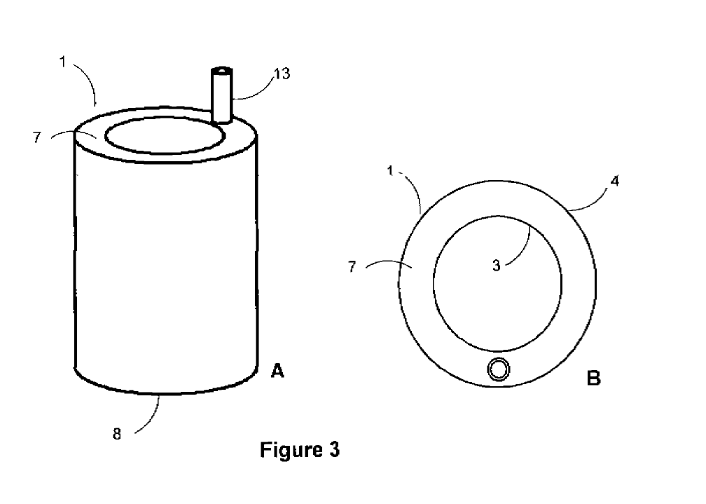

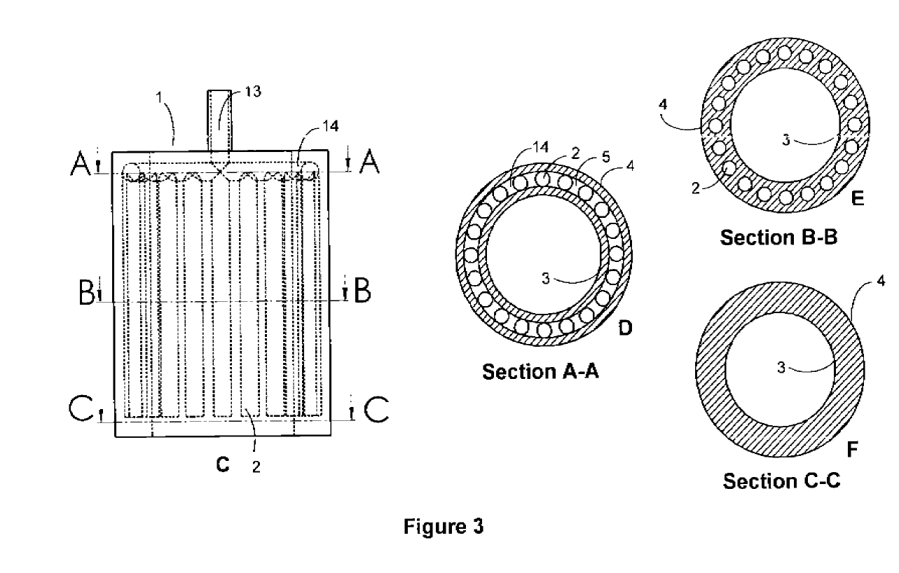

Referring now to FIGS. 3A and 3B, there is shown an inflatable device 1 wherein the inflation lumens 2 are in fluid communication with each other so that they may be inflated together. The inflation lumens 2 are inflated by introducing fluid pressure via an inflation port 13. In the embodiment the lumens 2 do not occupy the entire length of the device so that profiles of the lumens 2 are not visible at the front wall 7 or rear wall 8.

FIGS. 3C to 3F show the internal structure of the device 1. A connection conduit 14 is in fluid connection with each of the lumen 2 and the port 13 (as best seen from FIG. 3C and 3D). The connection conduit 14 is in the form of a ring which runs about one end of the annular body. The inflation lumens 2 are connected together so that the inflation lumens 2 are inflatable together.

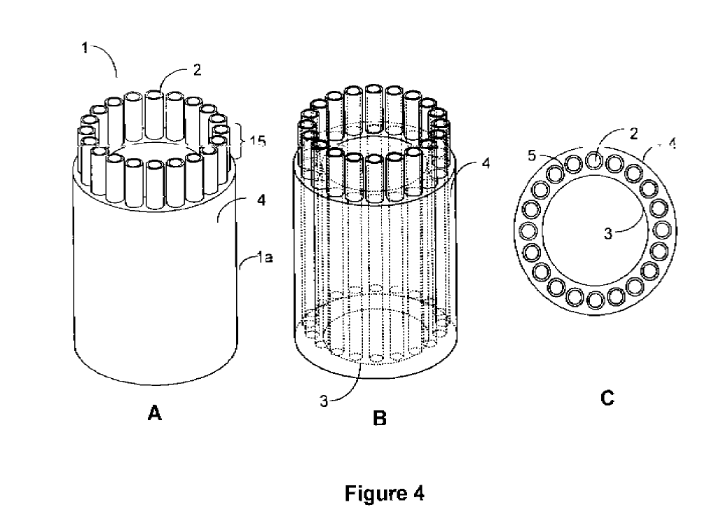

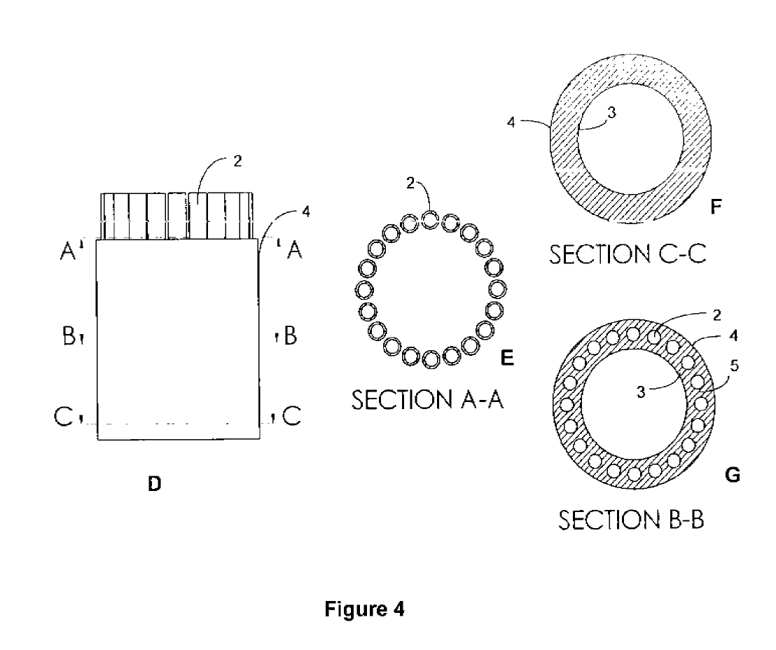

With reference to FIGS. 4A-4G, there is shown an alternate embodiment where the inflation lumens 2 are not in fluid communication with each other so that the inflation lumens 2 can be inflated separately. Each lumen 2 has a lumen-defining portion 15 which projects beyond the annular body 1a. The lumens are not interconnected so that they can be independently inflated. Independent inflation can allow more control of how expansion occurs and the shape of the device as it expands.

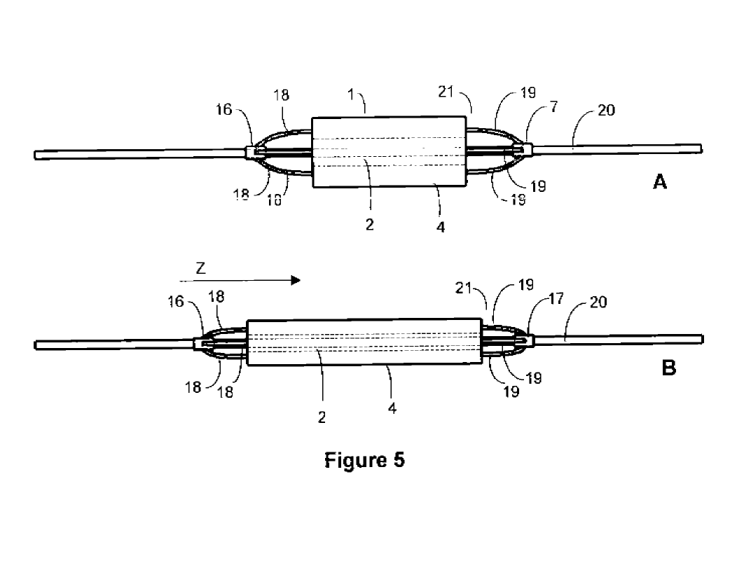

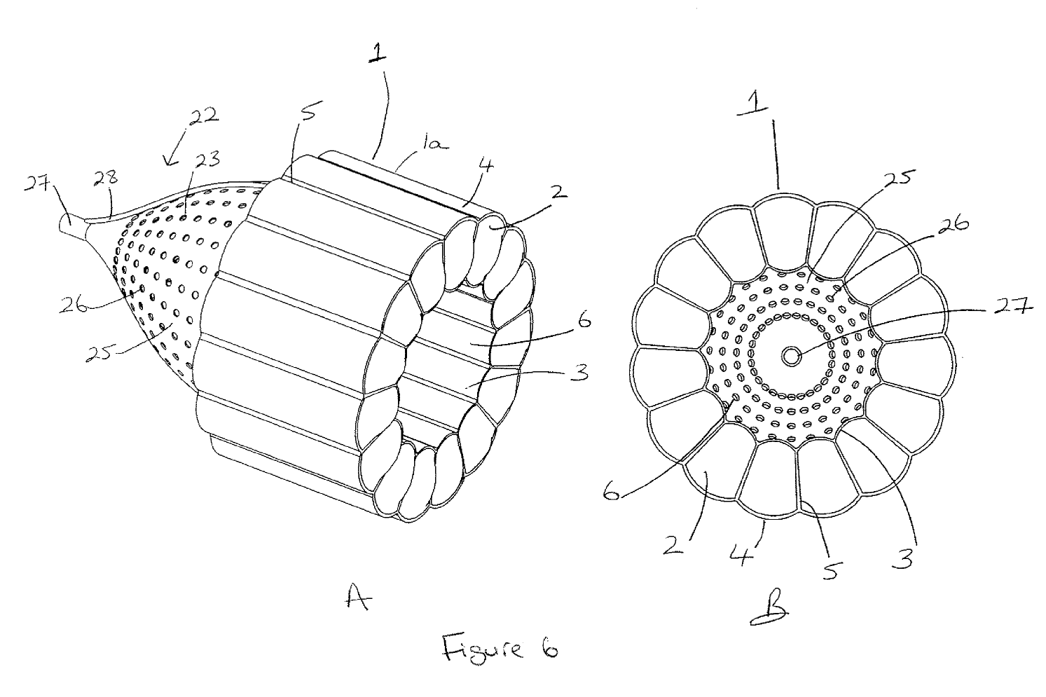

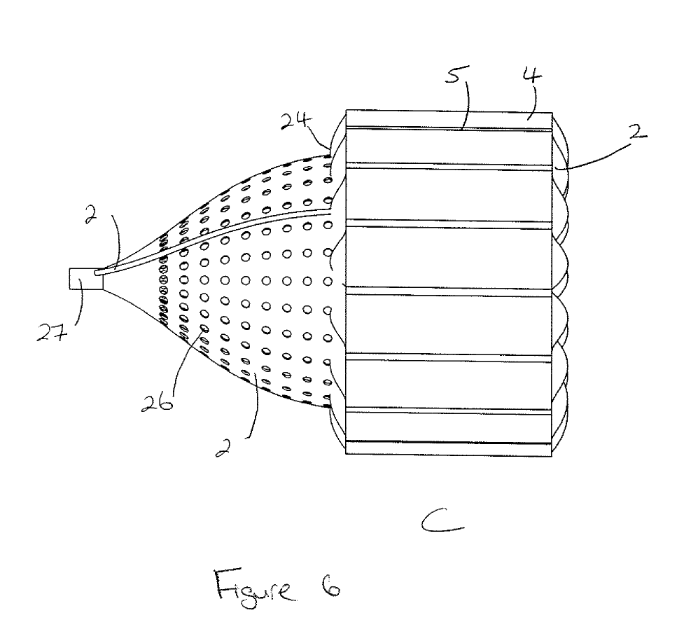

It will be appreciated that each device of the invention has an inflated, and a non-inflated configuration. Each device of the invention will also have a “rest” or “natural” configuration when it is uninflated. In other words the device will tend to a particular shape and size when it is not under any inflationary pressure. That shape and size will be smaller than that which is manifested upon exertion of inflationary pressure. It may be desirable nonetheless, to reduce the profile of the device for particular application for example insertion into the vasculature. Accordingly, it may be desirable to reduce the profile of the device to a profile which is smaller that that of the natural uninflated configuration. A configuration of the device suitable for such purposes is shown in FIGS. 5A and 5B.

FIGS. 5A and 5B show a device 1 of the invention mounted on a carrier which in the embodiment is a guide wire 20. A mechanism 21 is provided for reducing the profile of the device 1. In particular the mechanism allows a user to pull and/or twist the inflatable device 1 prior to and/or post deployment, in order to reduce the radial profile of the inflatable device 1. A proximal cuff 16 is attached to a first end of the inflatable device 1 via flexible connecting arms in the form of hollow tubing 18, while a distal cuff 17 is similarly attached thereto (at a second end) via flexible connecting arms in the form of hollow tubing 19. Inflation lumens 2 are shown in dashed outline.

Both the proximal cuff 16 and distal cuff 17 are mounted on a guide wire 20. In the present embodiment, the proximal cuff 18 is fixed to the guide wire 20, while the distal cuff 17 is slidably attached to the guide wire 20. To axially stretch the inflatable device 1 (from its rest position as shown in FIG. 5A) it is pulled proximally in the direction of the arrow Z utilising the proximal cuff 16. The device is thus stretched axially and the radial profile of the inflatable device 1 is lowered (as shown in FIG. 5B). It will be understood by those skilled in the art that the proximal cuff 16 may be slidably attached to the guide wire 20, while the distal cuff 17 may be fixed to the guide wire 20 so that pulling the distal cuff 17 distally may lower the radial height of the inflatable device 1. If desired both cuffs could be moveable to allow reduction of the radial profile. It will also be appreciated that release of the stretching forces will allow the device of the invention to return to its rest configuration. It will be appreciated that the mechanism of the invention can allow relative rotation of opposing ends of the device 1 to reduce the profile of the device by rotation of one end of the device relative to the other (twisting action).

A further embodiment of the present invention is to provide a device suitable for filtration applications such as embolic filtration. FIG. 6A-FIG. 6C show such a device. FIGS. 6A-6C show a device 1 which is similar in construction to that shown in earlier embodiments (such as in FIG. 1) with a filter attachment 22 attached to an annular body 1a. The device is shown in an expanded configuration deployed for use. Fluid, such as blood passing through the central lumen 6 is filtered by the attachment 22.

In the embodiment distal filtration is provided by the attachment 22, which takes the form of a filter basket 23. The mouth 24 of the filter basket is joined to the annular body 1a so that the filter opens up and out and collapses with the annular body. A filter mesh 25 comprising apertures 26 (such as micro-holes) filters fluid passing through. Debris or other undesirable particles in blood, for example debris created during removal of plaque etc from a vessel, will be caught by the filter basket 23. The filter mesh 25, which extends across the internal lumen 6 of the inflatable device 1, acts as an intraluminal embolic capture device to capture embolic material from blood passing through the internal lumen 6. The filter mesh 25 depicted in FIGS. 6A-6C is incorporated into a proximal end of the inflatable device 1. It may be secured to a carrier, for example a guide wire via a cuff 27. The filter mesh 25 may be constructed from a sheath of material which is the same as the material from which the annular body 1a is constructed. As such, one end of the inflatable device 1 with the filter mesh 25 is closed-off. When the annular body is collapsed to a rest state or is further reduced in profile, for example using a mechanism such as shown in FIG. 5, material caught by the filter is then trapped and will not fall out as the device is collapsed for removal following its deployment. The use of a compliant balloon ensures excellent vessel wall apposition for a range of vessel cross-sections, and as such, provides the invention with a competitive advantage over the more traditional nitinol-based filters, which have difficulty conforming to irregular shaped vessel, i.e. vessel with elliptical or asymmetrical shaped cross-sections. The elasticity of the balloon material is also utilised during deflation, to collapse the filter and trap any collected debris prior to retrieving the device. By collapsing the opening to the filter first (the proximal side of the balloon) the likelihood of debris escaping the filter can be minimised during balloon deflation.

In the embodiment the inflatable device 1 is inflated and collapsed by a pressure feed tubing 28. The pressure feed tubing 28 may be connected to a distal side of the device 1 which would ensure proximal collapse before distal collapse. By collapsing the proximal side of the inflatable device 1 the likelihood of debris escaping the filter mesh 25 can be minimised during deflation of the inflatable device 1. However, it will be understood by those skilled in the art that a number of conceivable variations may exist in relation to the number of guide wire attachment points or the specifics of the pressure feed system. For example, in tortuous vessels where guide wire positioning becomes more difficult it may be beneficial to reduce the number of attachment points between the filter and the guide wire, which may aid in avoiding improper placement.

It is understood that the filter may be constructed as a simple filter basket rather than as an extension of the inflatable device.

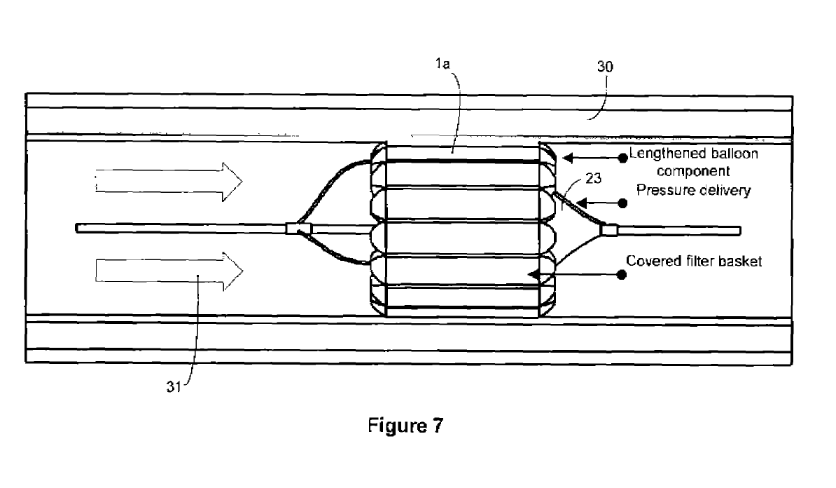

A variation of the filter devices of the invention is to house a substantial part of the filter within the annular body of the present invention. One way to achieve this is to increase the length of the multi-lumen inflatable device 1, thereby enveloping the filter basket. FIG. 7 shows such an embodiment of a device of the invention which includes a filter and which is generally similar in construction to that shown in FIG. 6.

The device is shown in a deployed state in a vessel 30. The arrows 31 indicate blood flow direction. In this embodiment the annular body 1a has been lengthened to accommodate a substantial portion of the filter attachment 22. The mouth 24 of the filter attachment is no longer attached to one end of the annular body 1a but is attached internally within lumen 6 to the inner wall 3. It is desirable that at least 10% such as at least 20%, for example at least 30% in general at least 40% of the (axial) length of the filter attachment is accommodated within the annular body.

Enveloping the filter basket 23 provides greater filter stability and vessel wall apposition. The risks associated with retrieving a device using a retrieval sheath is that the filter basket may become entangled for example with a stent which is deployed during an angioplasty procedure. However, as the filter basket and trapped debris will be safely contained beneath and within the device 1 during retrieval, such risks are ameliorated.

Alternatively, the trapped debris may be removed by suction, or initially physically broken up and removed by suction.

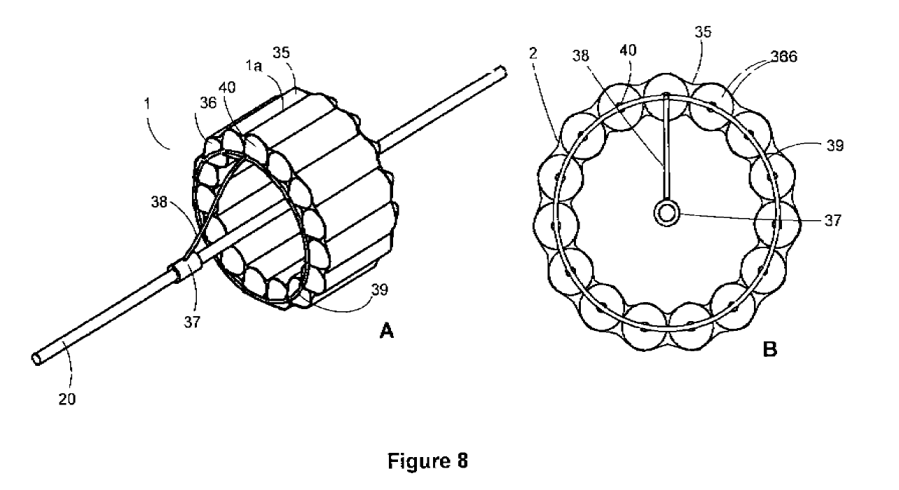

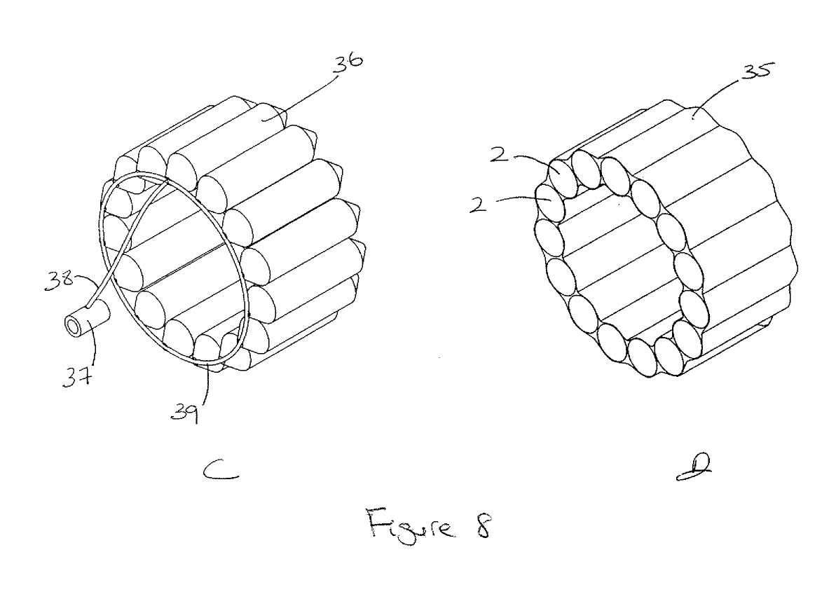

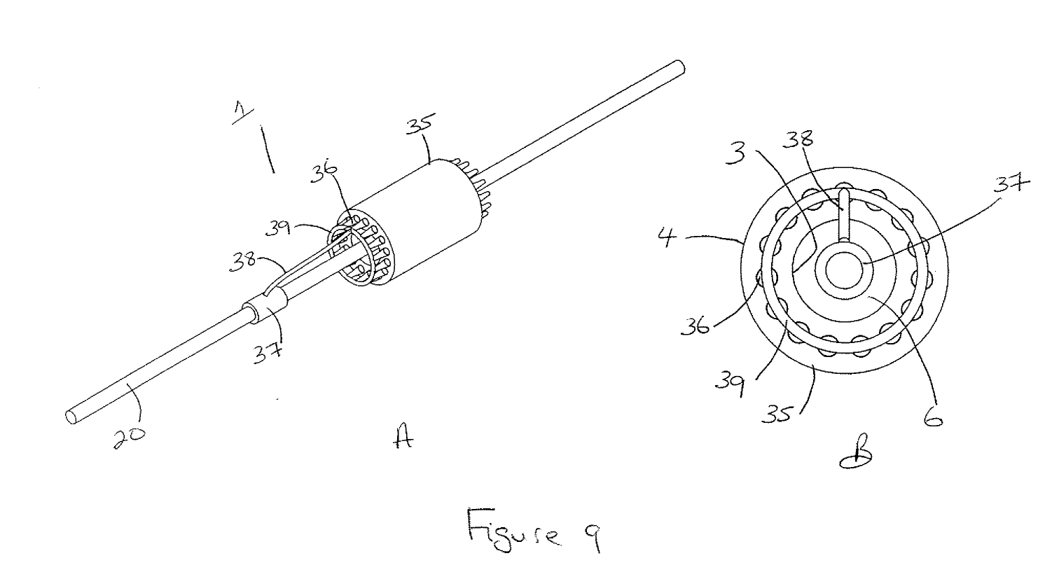

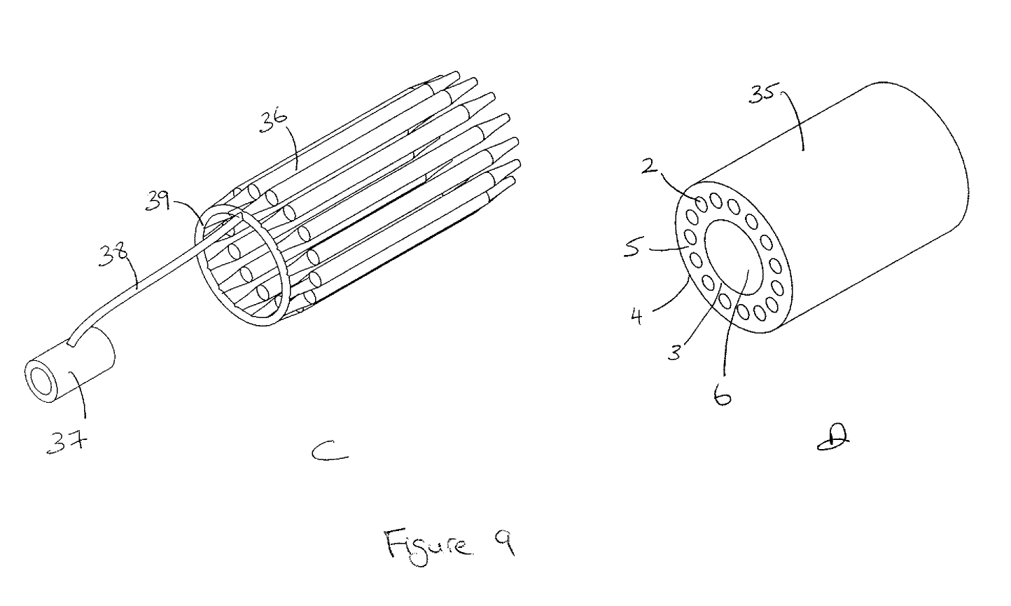

Referring now to FIGS. 8A-D and 9A-D, there is shown therein a further embodiment of the present invention wherein the inflatable device 1 comprises inflatable balloons which are insertable into an annular sheath. In this embodiment the inflatable annular body 1a comprises two separate components, an annular sheath 35 (best seen in FIG. 8D) and an annular series of balloons 36 (best seen in FIG. 8C) arranged to be accommodated within lumens 2 in the sheath 35.

In the embodiment, an annular arrangement of balloons which are desirably non-compliant balloons 36 composed of non-compliant material, are inserted into the inflatable lumens 2 to form the assembly of FIGS. 8A and B. The expansion of the inflatable lumens 2 is initiated through inflation of the non-compliant balloons 36. Desirably the sheath 35 is elastic. It will thus function to bias the device towards an unexpanded configuration and to collapse any filter attachment as described above. When subjected to inflationary pressure it will form the desired annular structure. As with compliant balloons the non-compliant balloons can have any desired cross-sectional shape, for example they may be oval or crescent shaped. It will be appreciated that an optimal cross-sectional shape can be applied for the task in hand.

FIGS. 8A and 8B demonstrate the inflatable device 1 when in an inflated state. The inflatable device 1 is anchored to a guide wire 20 by a distal cuff 37. The annular body 1a is attached to the cuff 37 by a flexible arm 38, which in the embodiment also acts as a pressure feed tube. A ring tube 39 has two functions, a first function is to act as a mount for the balloons 36 and to hold them in a suitable configuration for mating with the lumens of the sheath 35. A second function is to provide inflationary pressure for the balloons 36. A nose 40 on the end of each balloon is in fluid communication with the ring tube 39. The ring tube 39 acts as a conduit for inflationary fluid from the arm 38 to the balloons 36. The ring tube 39 and the arm 38 are flexible and allow the device to collapse.

The use of non-compliant balloons 36 within the elastic sheath 35 allows for greater control during device deployment, enabling devices to be custom produced to fit specific vessel sizes.

FIGS. 9A-9D respectively show equivalent views of each of FIGS. 8A-8D save that the device is in the deflated state in each of FIGS. 9A-9D.

It will be understood by those skilled in the art that a number of conceivable variations may exist in relation to the number of guide wire attachment points or the specifics of the pressure feed system in the above-described embodiment.

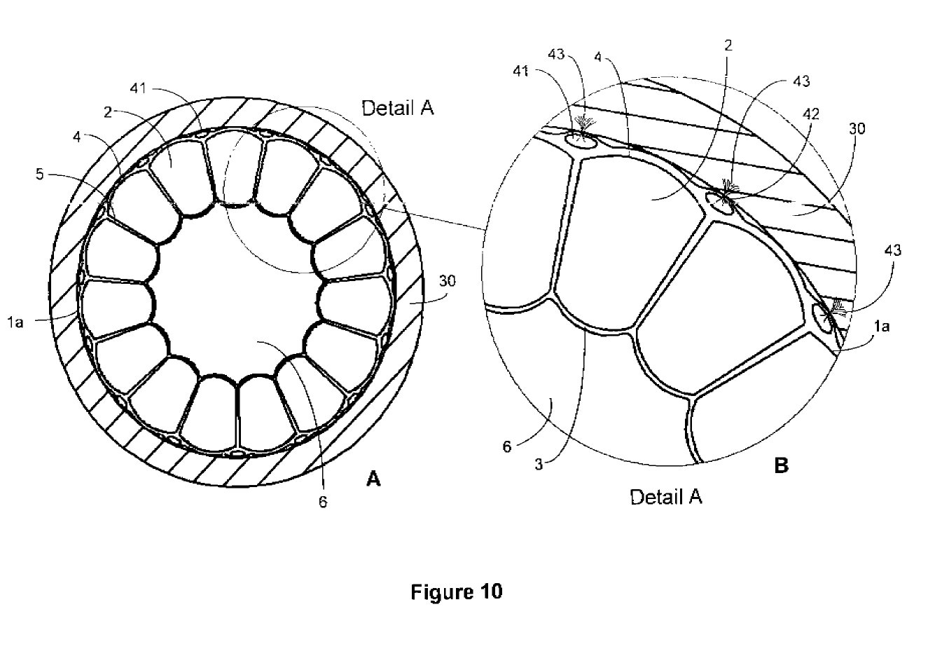

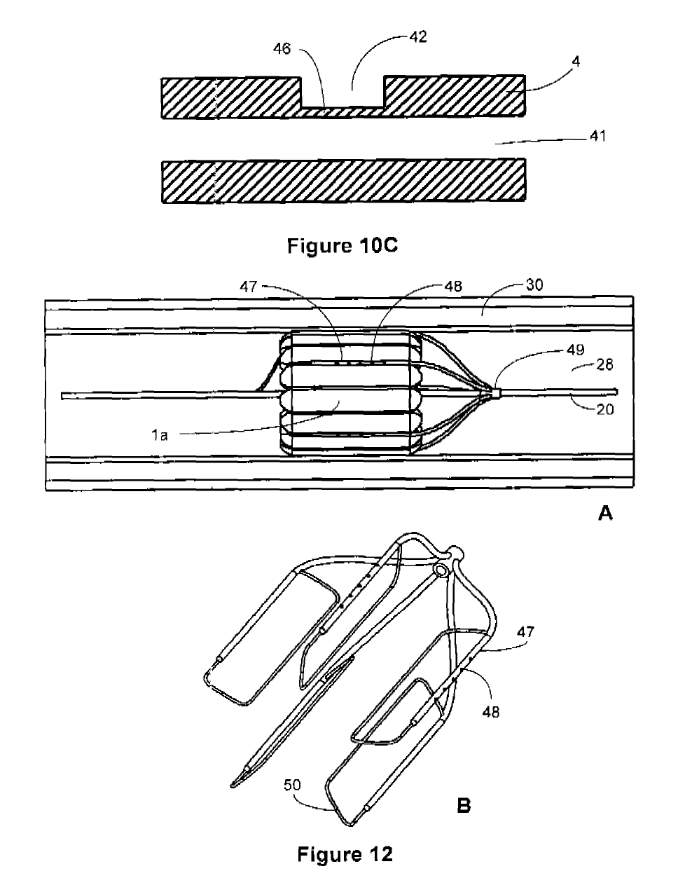

Referring now to FIGS. 10A and 10B there is shown therein a further embodiment of the present invention wherein the inflatable device 1 comprises infusion means for infusing a (therapeutic) substance into a vessel. In particular the device is adapted to infuse the material when a predetermined inflation pressure is reached.

As can be seen from the Figures the device is provided with a plurality of rupturable reservoirs which in the embodiment are a series of infusion tubes or lumen 41 disposed about the periphery of the annular body 1a. In the embodiment the device is deployed (expanded) within a vessel such as a blood vessel 30. In the embodiment a rupturable reservoir is provided on the periphery of the device and between each adjacent pair of inflation lumen 2. This allows for the even distribution of the therapeutic material to the vessel 30.

The infusion lumen 41 are provided as an integral part of the annular device.

In particular they form an integral part of outer wall 4 of the device. The location of the infusion lumen 41 is chosen so as to be close to the vessel 30 following inflation. Numerous micro-holes 42 (best seen in the enlarged view of FIG. 10B) in the outer surface of the outer walls 4 of the annular balloon component (coinciding with the infusion lumens 41) enables a therapeutic material 43 contained within the infusion lumens 41 to be infused into the vessel following deployment. The forced collapse of the infusion lumen 41 resulting from annular balloon component expansion combined with the compression of the annular balloon component against the vessel wall 30 ensures full evacuation of the therapeutic material 43.

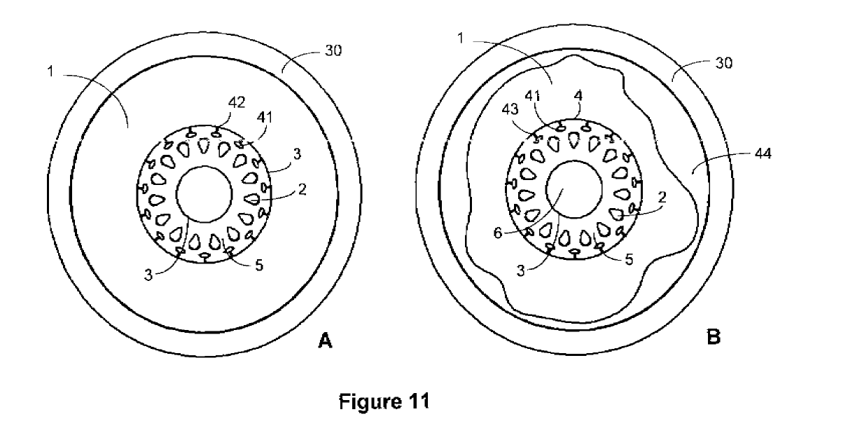

FIGS. 11A and 11B show the embodiment of FIG. 10A and 10B before inflation and within the vessel 30. In FIG. 11A the vessel 30 is a vessel free of plaque whereas in FIG. 11 B vessel 30 is shown as a diseased vessel with plaque 44 partially obstructing the vessel. When the device of the invention is a compliant one it will be able to take up the internal shape of both the non-diseased and diseased vessels.

A variation of this embodiment may be to include intentional failure points as shown in the schematic representation of FIG. 10C. In that embodiment a rupturable member 46 which functions to occlude the micro-holes 42 of the infusion lumens 41, thereby preventing the early ill-timed release of the therapeutic compound 43 and allowing for greater control over the process of drug release. The failure members may be designed to fail at a pre-determined level of expansion, so that ideally, drug release only occurs once contact has been established with the arterial wall.

A further variation of an infusion device of the invention is instead of having the infusion means as a rupturable part of the annular body, providing for delivery of the material to be infused by other means. In particular it is possible to provide one or more infusion tubes, which can deliver a therapeutic fluid. For example it is possible to house tubing within the lumen of the annular balloon component. A perceived benefit of this approach is that the therapeutic material may be injected along the length of a catheter system and as such is physician-controlled (unlike the embodiments where pressure failure is used). Assembly of the device may involve an over-moulding step to encase tubing in the annular body or alternatively, the tubing may be attached to the annular balloon component thereof.

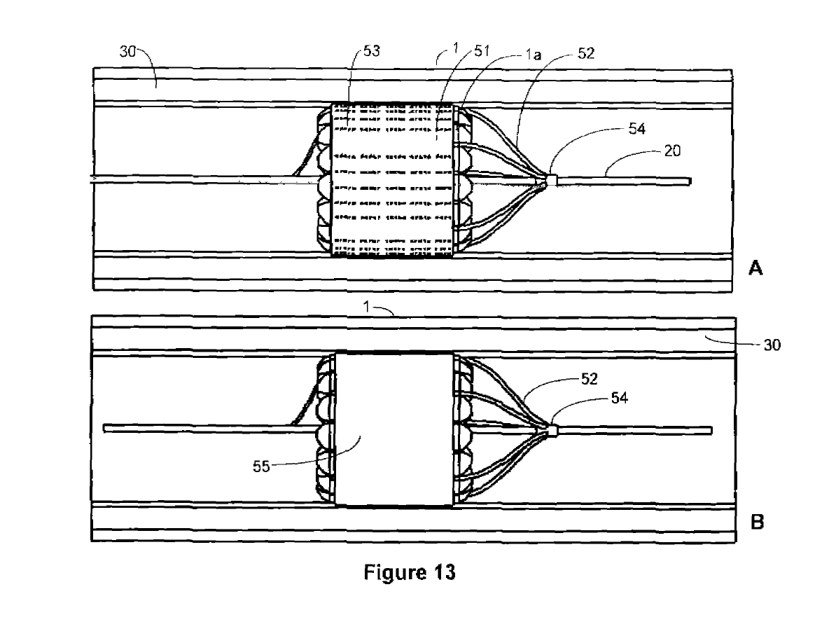

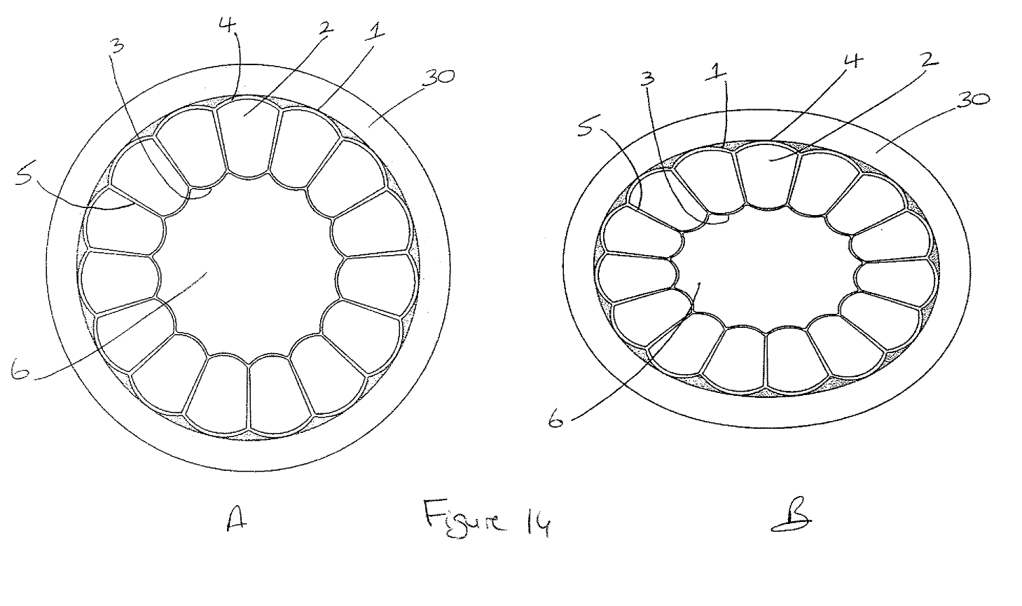

FIGS. 12A, 12B, 13A and 13B show embodiments of such a device. The embodiment of FIG. 12 A is similar to that of FIG. 5 with the annular body 1a shown in the inflated condition with a vessel 30. The annular body 1 is attached to a guide wire 20 by a series of arms 47 which form a cage which receive the annular body. The arms 47 are attached to the guide wire 20 via a cuff 49. The arms 47 function as conduits and are adapted to receive a therapeutic fluid from a therapeutic fluid source. Suitable apertures 48 in the arms 47 allow therapeutic materials to be ejected into the vessel 30 once delivered through arms 47. This embodiment allows numerous arms 47 surrounding the annular balloon component structure to be utilised. As with the previous embodiment, infusion tubing may provide fluid communication through the guide wire lumen and along the length of a catheter, thereby enabling the operator to fully control the compound delivery. The manner in which the infusion tubes are connected to the annular balloon of the inflatable device 1 must not inhibit the expansion of the annular balloon component. To ensure this, elastic cuffs 50 (best seen in FIG. 12B) are used to secure the annular balloon to the arms. The elasticity of the cuffs 50 should match that of the annular balloon so that a firm connection is maintained whether the annular balloon component is in its inflated or deflated configuration.

An alternative embodiment may include the use of a sheath 51 over the annular balloon component 1a of the inflatable device 1. The sheath 51 does not impede inflation or deflation of the device and is desirably elastic. In the embodiment the sheath 51 forms a sealed cavity spanning the circumference of the inflatable device 1. (If desired, that cavity can be partitioned into separate infusion lumen) To accomplish this, the sheath 51 may be bonded to the proximal and distal circumference of the annular balloon component of the inflatable device 1 in order to create a seal. Arms 52 (which are similar to other embodiments) attach the device 1 to (a cuff 54 which in turn attaches the device to) a guide wire 20. In the embodiment the device is shown in an expanded configuration within a vessel 30.

The arms 52 form conduits for the delivery of therapeutic fluids to the cavity between the sheath 51 and the device 1a. The therapeutic compound material thus pools within the cavity. Numerous perforations 53 on the surface of the sheath 51 allow for infusion of the therapeutic compound into the contacting vessel wall 30. A benefit of this embodiment is that the entire circumference of the vessel can be treated without the need for repositioning the device 1. Like those other embodiments that incorporate infusion tubing, the compound delivery is physician controlled.

A variation of this concept is shown in FIG. 13B where there is shown a very similar arrangement to FIG. 13A. In the FIG. 13B embodiment there is provided a porous sheath 55. The porosity of the sheath 55 is sufficient to allow infusion of therapeutic fluid. As with the embodiment of FIG. 13A, a cavity between the annular balloon component and the sheath is supplied with therapeutic fluid by arms 54. The cavity collects the therapeutic material and releases it at a desired rate by way of capillary action through the thickness of the sheath 55. Therapeutic material is thus released around the circumference of the device 1. In doing so, less of the material will be taken away by the blood stream and this increases the likelihood that successful infusion to the vessel wall will occur. The porous sheath 55 must expand along with the annular balloon component and as such, a suitable material such as an elastomeric foam may be required.

FIGS. 14A and 14B demonstrate the apposition of the inflatable device 1 of the present invention in those vessels which are substantially circular or non-circular in cross-sectional shape. The level of conformity achieved with the inflatable device 1 of the present invention is exemplary, particularly in non-circular shaped vessels. In particular in FIG. 14B it is shown that a device of the invention can provide very good vessel wall contact even in vessels that are non-circular for example substantially elliptical in cross section. The fact that the device of the invention comprises an inflated annular cylinder allows it to adapt to varying vessel shapes.

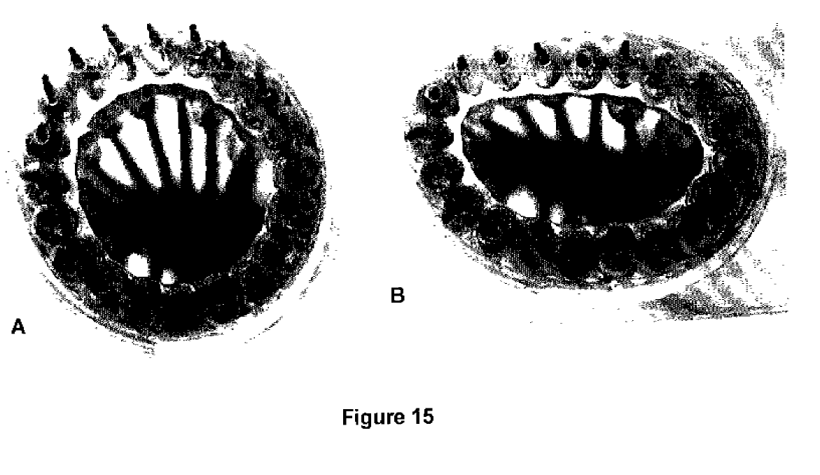

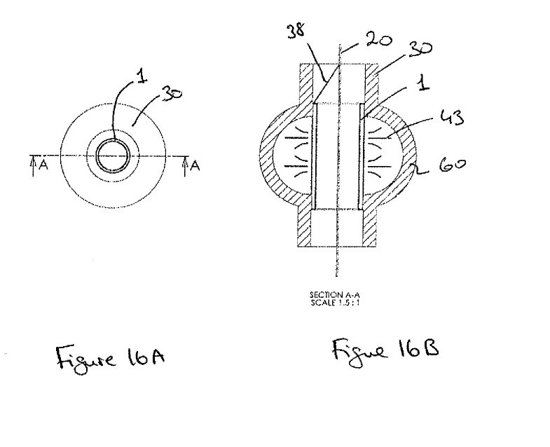

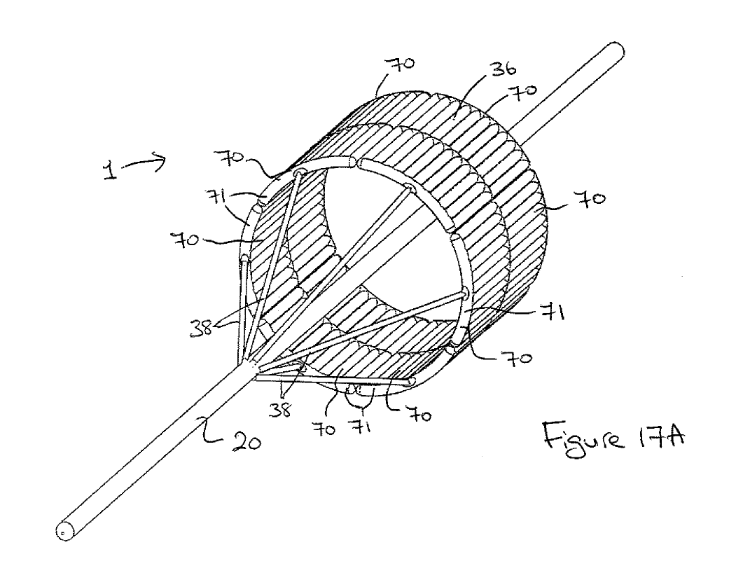

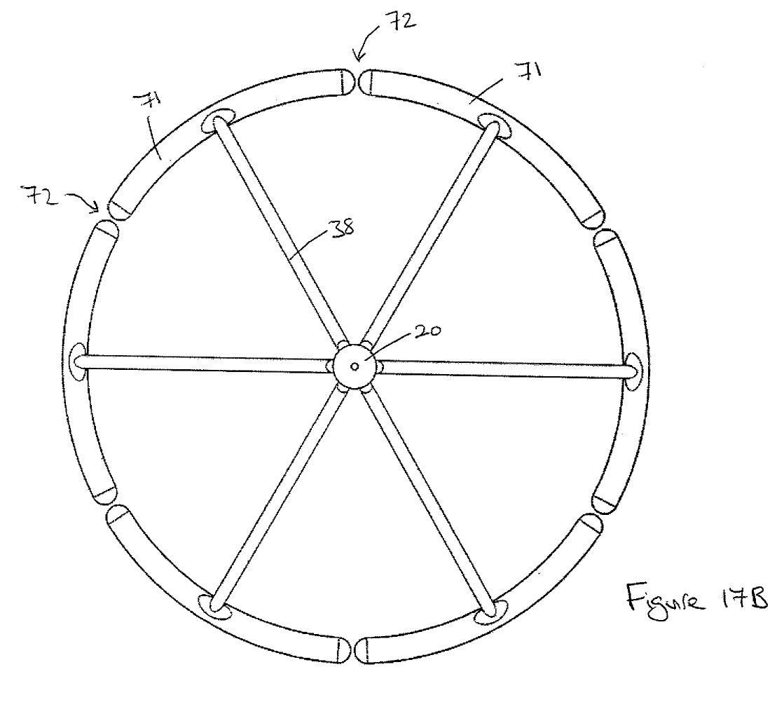

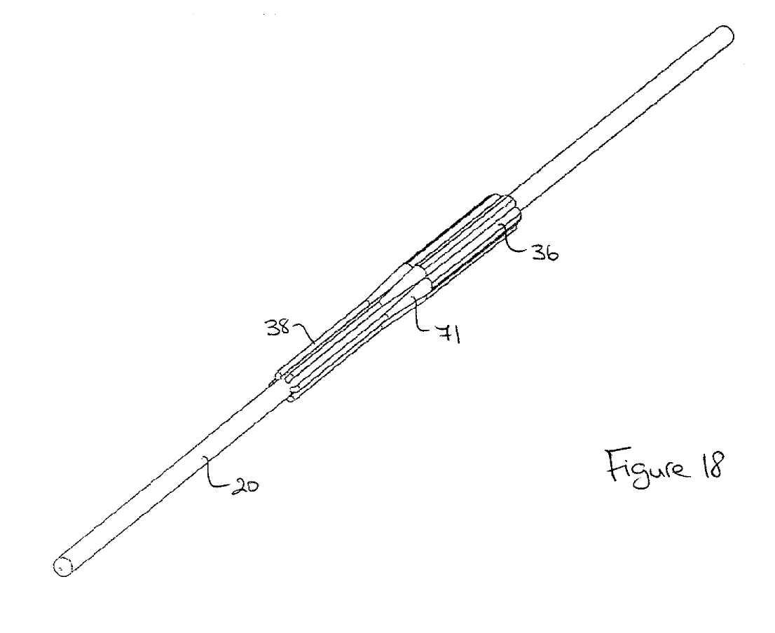

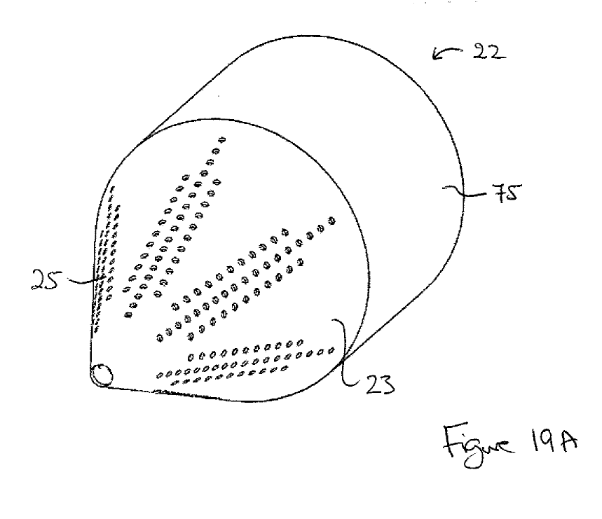

FIGS. 15A and 15B show photographic images of a device 1 of the invention that was constructed as follows: An elastic sheath with a number of lumen defined therein was moulded from silicone material. Non-compliant balloons were then inserted into the lumens.

As can be seen from FIGS. 15A and 15B, the device when moved to an inflated configuration conforms very well to the shape of the vessel in which it is place.

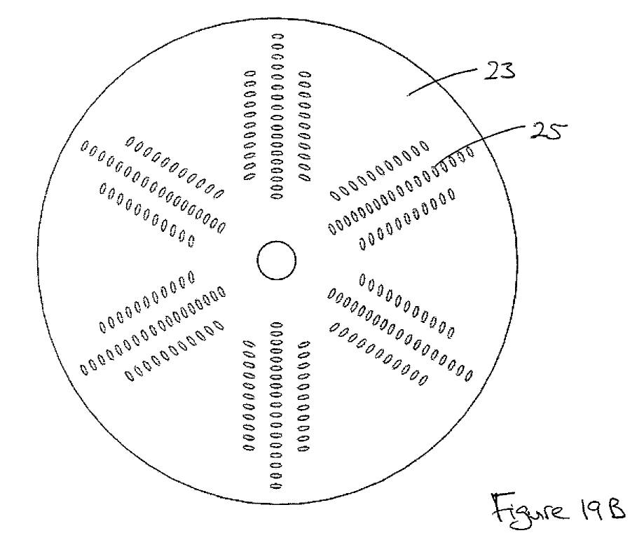

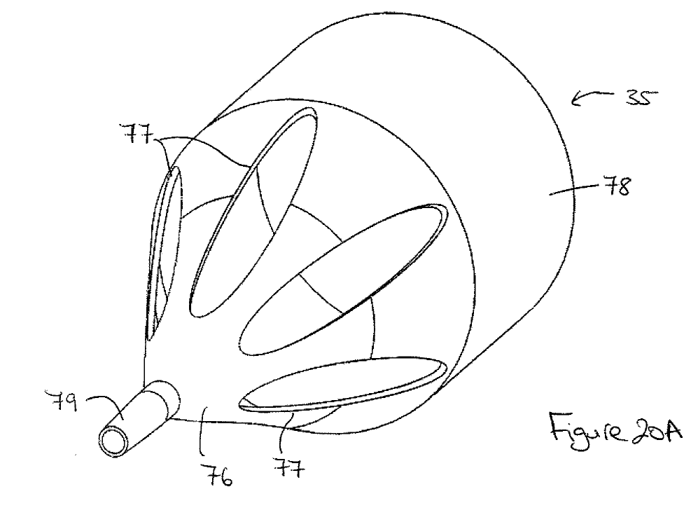

FIGS. 16A and 16B show respective cross-sectional views of a device 1 of the invention in situ within a vessel 30. The vessel 30 has a target site which is a bulging of the vessel caused by an aneurism 60. The device 1 is of the same construction as that shown in earlier Figures such as FIG. 10. It is adapted to infuse a therapeutic material 43 which may be a clotting agent or other suitable agent for treating the aneurism. It will be appreciated that upon expansion (as shown in FIG. 16B) the device 1 of the invention will abut and closely mate to the vessel 30. This means infused therapeutic material is protected from being carried away from the target site by blood flow. Residence time of the therapeutic material is increased thus also increasing the therapeutic effect.

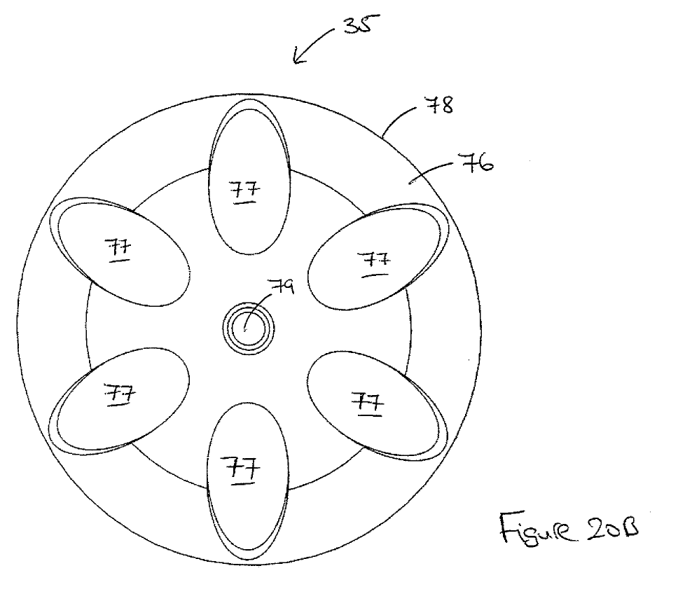

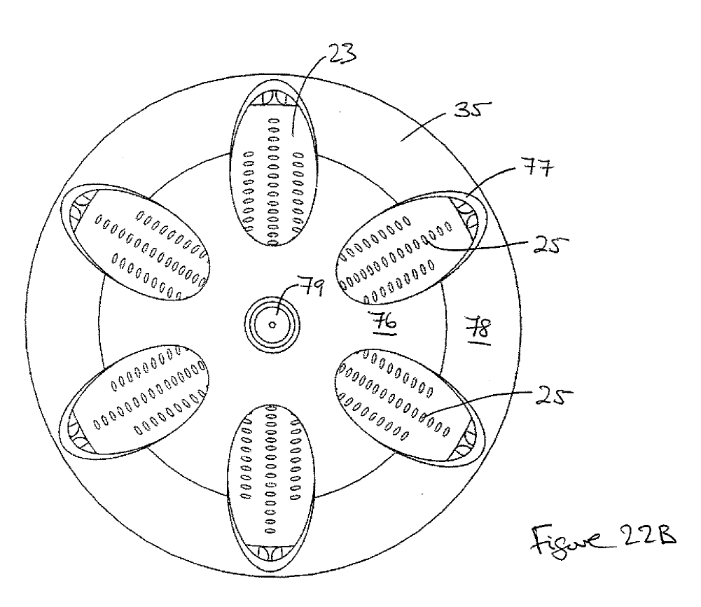

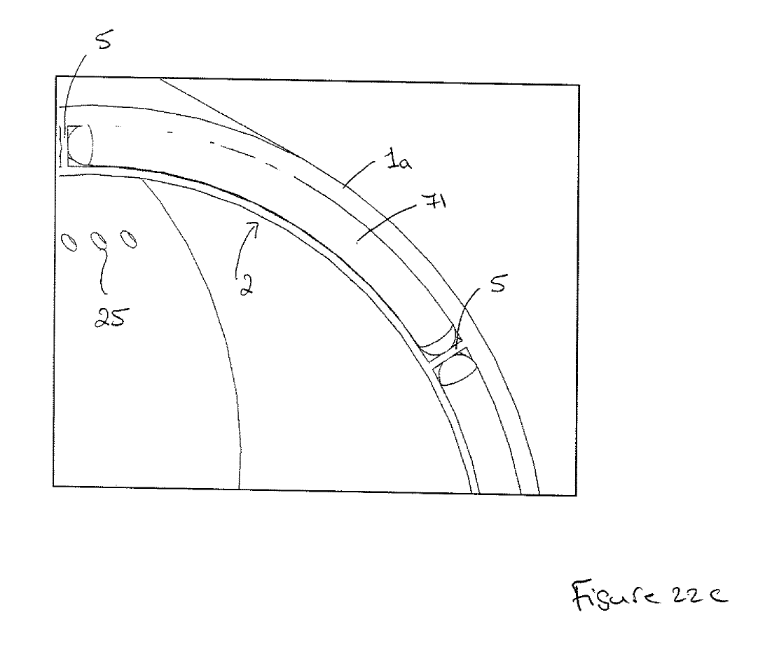

Referring now to FIGS. 17A-B and 9A-D, there is shown therein an embodiment of the present invention similar to that shown in FIGS. 8A and 8B wherein the inflatable device 1 comprises inflatable balloons which are insertable into an annular sheath 35 (best seen in FIG. 20A). In this embodiment the inflatable annular body 1a comprises two separate components, the annular sheath 35 and an annular series of balloons 36 (best seen in FIG. 17A) arranged to be accommodated within lumens 2 in the sheath 35.

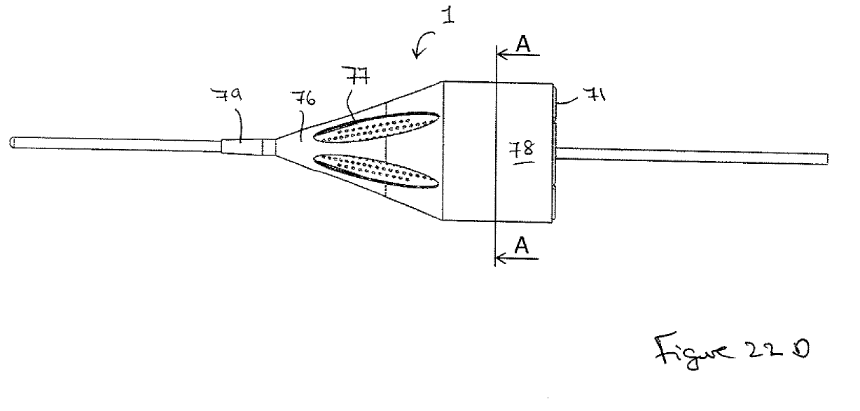

In the embodiment, an annular arrangement of balloons which are desirably non-compliant balloons 36 composed of non-compliant material, are inserted into the inflatable lumens 2 to form the assembly of FIG. 21A. The expansion of the inflatable lumens 2 is initiated through inflation of the non-compliant balloons 36. Desirably the sheath 35 is elastic. It will thus function to bias the device towards an unexpanded configuration and to collapse a filter attachment 22 as described below.

In FIG. 17A and 17B it can be seen that the annular arrangement of balloons is made up of a plurality of non-compliant balloons that are provided in groups 70 each being in an arc shape and thus forming a segment of the annular arrangement. Together the groups 70 form an annular arrangement. In the embodiment of two series of groups 70 (placed end to end) are provided to form the overall annular shape. Each of the balloons in groups 70 can be inflated at one time being connected by an end manifold tube 71. Each manifold tube 71 is supplied fluid by a tube 38 which in turn is carried on a guidewire/catheter tube 20. Inflationary fluid can be supplied through guidewire 20 into respective tubes 38 to each manifold 71 inflating all of the balloons 36 in each segment. When deflation is required the fluid is removed in a reversal of that process. As best seen from FIG. 17B the segments 71 are not joined together being separate parts in a circumferential direction. This means that each pair of adjacent groups has a space 72 therebetween. This allows the annular arrangement of balloons to be inserted into a sheath 35 with respective groups of balloons in respective pockets or lumen.

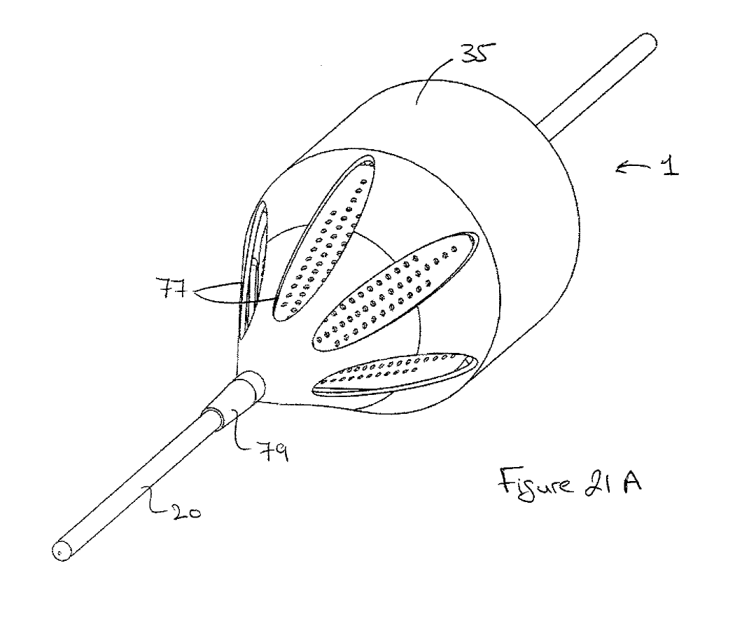

FIG. 18 shows the device of FIGS. 17A and 17B in a collapsed condition in FIG. 18. As will be appreciated the device of the invention has a very low profile when in the collapsed condition allowing it to be inserted into small vessels in the body, for example in peripheral vessels.

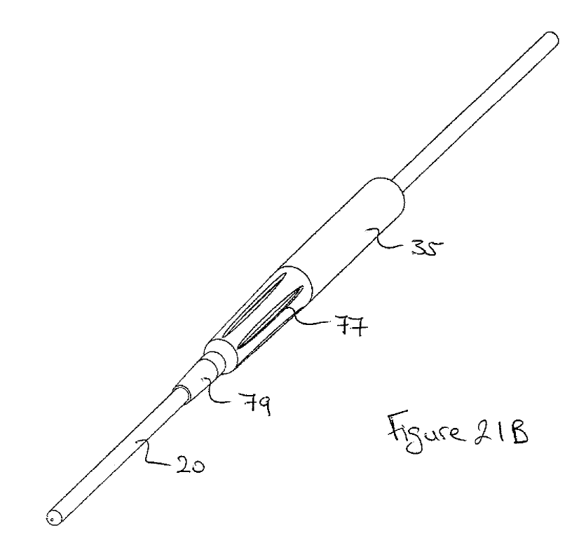

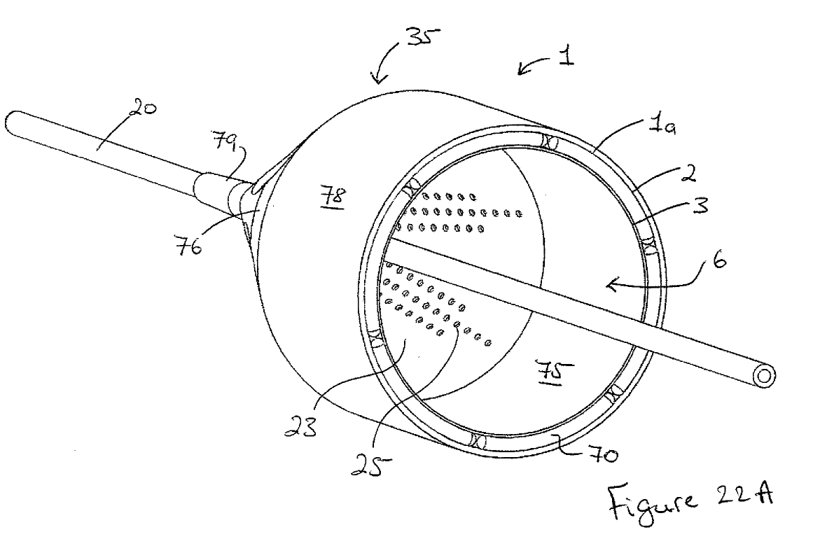

A filter attachment 22 suitable for use with the device of FIGS. 17 and 18 is shown in perspective view in FIG. 19A and end view in FIG. 19B. The device comprises a substantially conical part which forms a filter basket 23. Apertures defined in the basket 23 define a filter mesh for catching embolic materials while allowing blood flow. A substantially cylindrical skirt 75 on the filter attachment is suitable for attaching the attachment 22 to the device of FIGS. 17 and 18.

FIG. 20A and FIG. 20B respectively show a perspective view and an end view of a sheath 35 suitable for use with the device of FIGS. 17 and 18 and the filter attachment of FIGS. 19A and 19B. The sheath 35 comprises a substantially conically shaped portion 76 with elongate substantially elliptical apertures 77 defined therein. The conically shaped portion 76 is attached to a substantially cylindrical skirt 78. A nose piece 79 on the sheath 35 allows for attachment of the sheath to a guidewire/catheter tube 20.

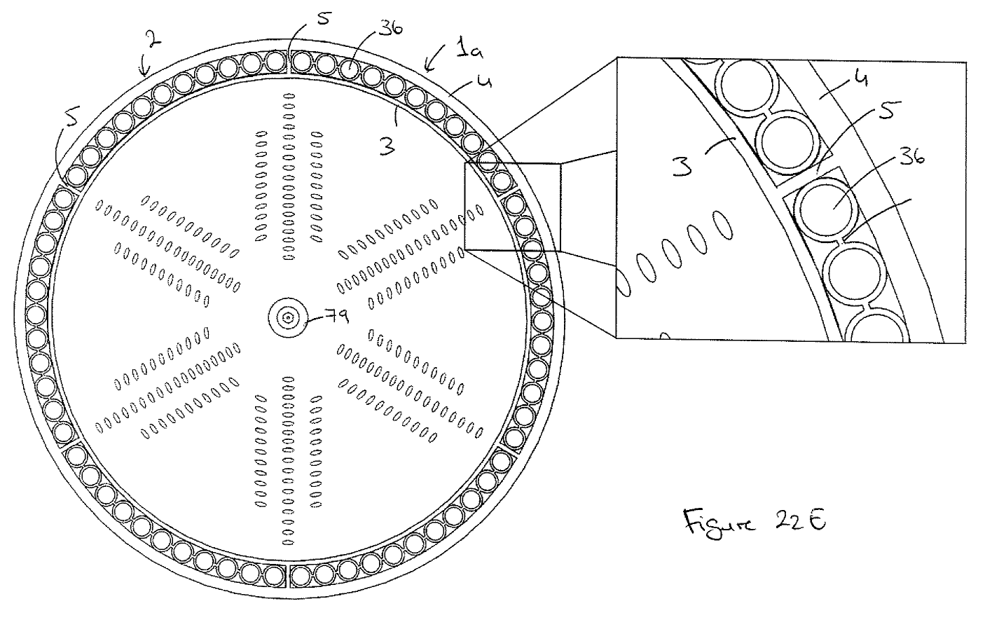

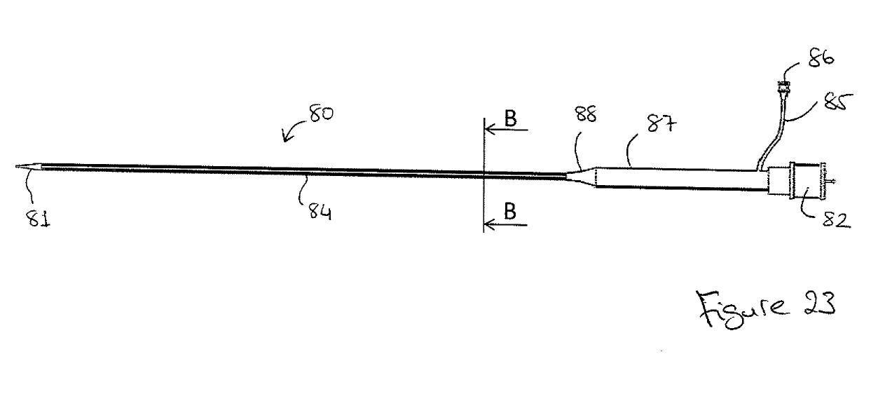

The annular arrangement of balloons of FIG. 17A/17B; the filter attachment 22 of FIG. 19A/19B and the filter basket 23 of FIG. 20A/20B are assembled as shown in FIGS. 21A, 21B, 22A, 22 B 22C, 22D and 22E. FIG. 22A FIG. 22B and FIG. 22D show, respectively from a rear perspective, front plan and side elevation view the assembled device of FIGS. 21A/21B in an expanded configuration.

In particular the balloons 36 have been inserted into pockets or inflation lumen 2 defined in the annular body 1a of the device. The pockets 2 are defined by partition walls 5 as best seen from the enlarged view of FIG. 22C. The filter attachment 22 has been attached to the annular body 1a with the substantially conical part forming a filter basket 23 and with the nose piece 79 on the guide wire 20. The skirt 75 on the filter attachment 22 has been inserted into the inner (blood flow) lumen 6 of the device and attached to the inner annular wall 3 of the annular body 1a. The sheath 35 is then overfitted (as best seen in FIG. 22B) over the annular body/filter attachment with the skirt 78 of the sheath 35 attached to the external annular wall 4 of the annular body. The substantially conically shaped portion 76 of the sheath 35 overlies the conical portion 75 on the filter attachment. The filter mesh apertures 25 defined in the basket 23 for catching embolic materials while allowing blood flow with elongate substantially align with elongate elliptical apertures 77 of the sheath 35. As best seen from FIG. 21A the device (in its expanded configuration) can be used as an embolic filter as described for other embodiments above. When collapsed the device takes the form shown in FIG. 21B where it is has a very low profile making it suitable for use in smaller vessels.

As best seen from FIG. 22e, and in particular the inset enlarged view of FIG. 22e, the annular body 1a has inner and out annular walls 3,4 with partition walls 5 defining inflation lumen 2. In total there are six (6) inflation lumen 2 each comprising a group 70 of interconnected balloons 36. There are 12 balloons 36 in each group. As is desirable in all embodiments the balloons 36 in a given inflation lumen are interconnected sided-by-side. In the embodiment the interconnection is formed by connecting linkers 74. The groups 70 of balloons are interconnected to each other as best seen from FIGS. 17A and 17B.

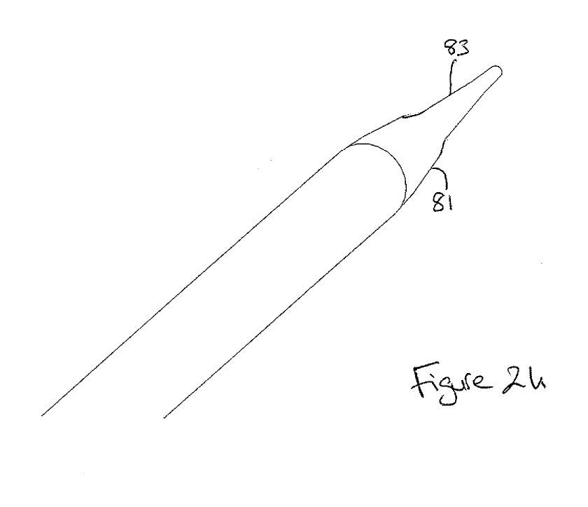

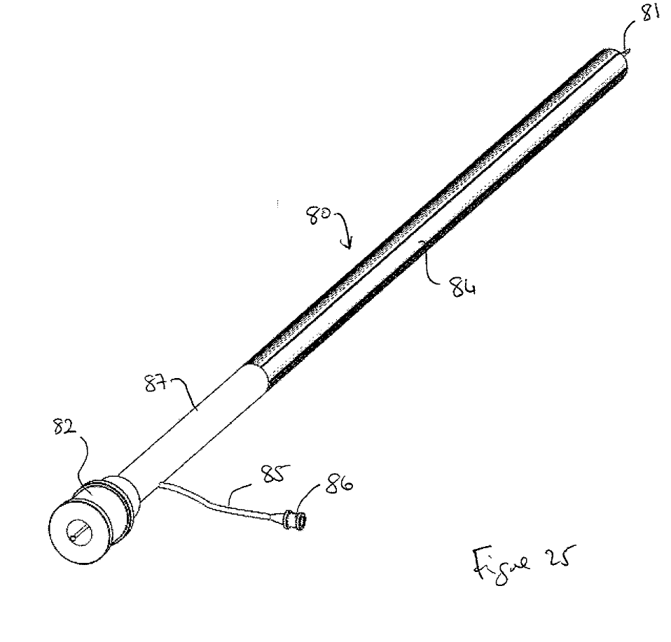

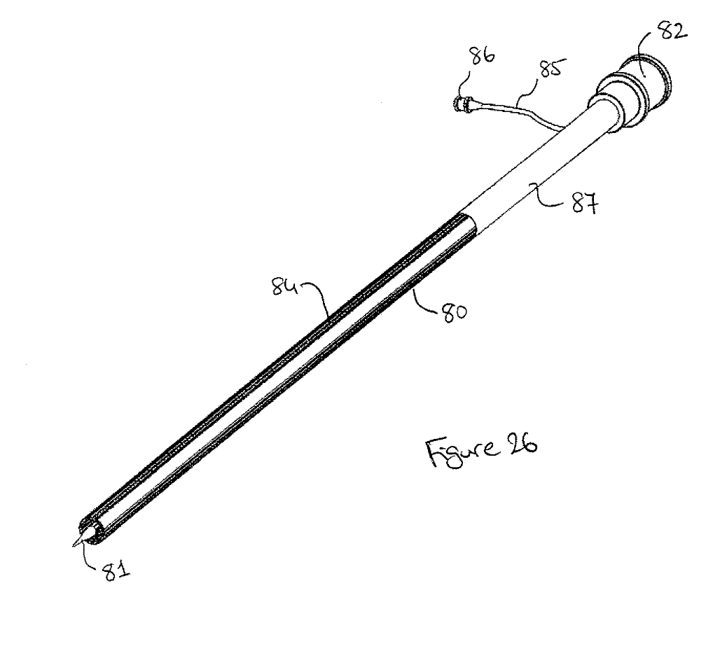

FIG. 23 is a perspective view, in an unexpanded configuration, of a device of the invention in the form of an introducer or dilation catheter 80, with a catheter body 84 an extendable/retractable tip 21 at a proximal end thereof. The catheter is used for introducing other devices into the vasculature for example heart valves, heart valve repair devices and aortic repair devices. The catheter 80 includes an inflatable device according to the present invention which is best seen from FIGS. 25-28. The catheter 80 includes (at a distal end) an adapter 82 which may include an introducer valve and/or luer lock fitting. The tip 81 has a nose piece 83 (see the enlarged view of FIG. 24) which can be advanced and retracted. When advanced it can be used to dilate a vessel in which it is placed as it advances. This allows the catheter body 84 to be advanced. This is particularly of use in smaller vessels. In the embodiment shown the catheter body 84 can be expanded. An inflation tube 85 with an appropriate valve fitting 86 can be use to inflate or deflate the catheter body 84. The catheter further comprises a non-inflatable portion or plug 87 which is insertable into a vessel and which has a tapered nose 88. The non-inflatable portion 87 will generally be dimensioned to have the same dimensions as the catheter body when the catheter body 84 is inflated. The non-inflatable portion will sufficiently occlude the vessel at the point of entry to prevent blood loss past the device once inserted.

FIG. 25 is a perspective view, in an expanded configuration, of the device of FIG. 23. As can be seen from a comparison of FIGS. 25 and 23 the device is substantially greater in cross sectional dimension (for example diameter) when it is in its expanded configuration. When in its expanded configuration it can be used to dilate vessels. It is advanced when in the collapsed (FIG. 23) configuration and can be advanced as far is desired into a vessel. When in position it is expanded by introduction of a pressurised fluid using the inflation tube 85. In the expanded configuration it can be used for delivery of a device such as a valve into the appropriate location.

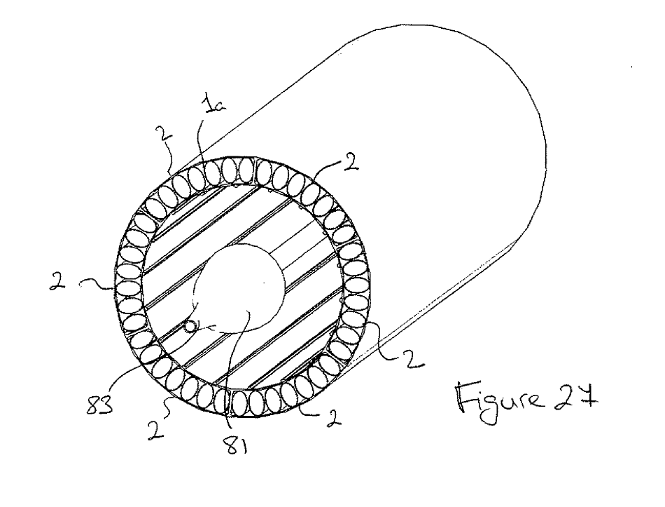

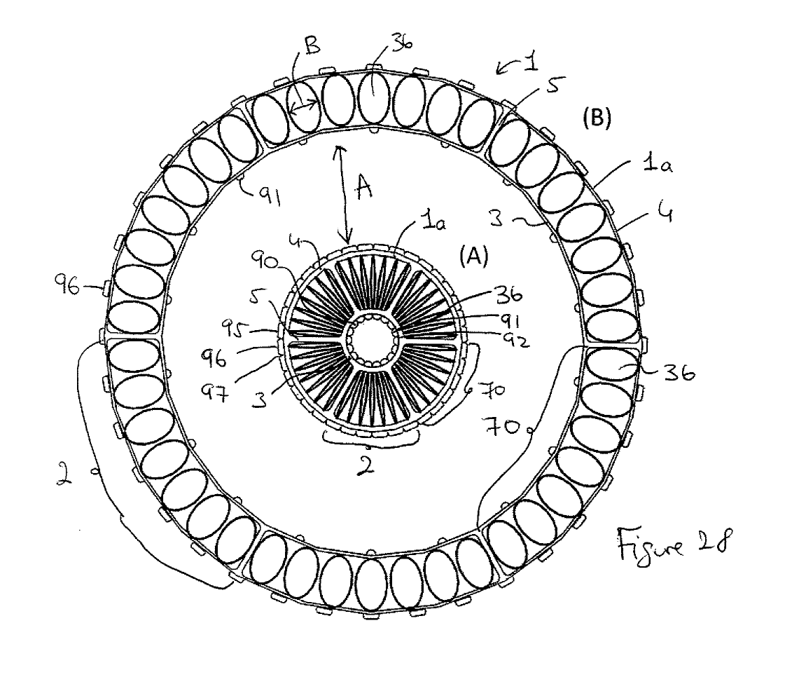

FIG. 26 is a perspective view from the proximal end, in an expanded configuration, of the device 1 of FIG. 23. The catheter is an inflatable device for use within the vasculature of a body. It has an expandable annular body 1a which is expandable by inflation of a series of inflation lumen 2 (in the embodiment 6 inflation lumen 2) defined therein. This is best seen from FIG. 27 which is an enlarged view of the proximal end of the device in an expanded configuration with the tapered tip retracted and from FIG. 28 which gives cross-sectional views in both the unexpanded and expanded configurations denoted by (A) and (B) respectively. Within the inflation lumen 2 are groups 70 of (interconnected) non-compliant balloons 36 (7 balloons in each group). The balloons 36 are arranged in an arrangement similar to that shown in FIG. 17A with each group being in an arc shape within their respective lumen 2 and thus forming a segment of the annular arrangement. Together the groups 70 form an annular arrangement. Each of the balloons in groups 70 can be inflated at one time.

FIG. 28 shows in cross-sectional view inflated (the external ring indicated by (B) in the drawing) and deflated (the inner ring indicated by (A) in the drawing) states. Inflation (and deflation) moves the device between the two states as indicated by arrow A. As is desirable a plurality of non-compliant or semi-compliant balloons 36 are provided in each of at least two lumen and in particular are provided in each lumen. Inflation of the device is achieved by inflating the non-compliant or semi-compliant balloons 36.

In the non-inflated state of the device, the balloons 36 are substantially flat or planar. They are arranged side-by-side in a concertina or accordion-type arrangement with the plane of each flat balloon arranged in a substantially radial direction relative to the annular body 1a. The balloons 36 are arranged to expand upon inflation in a direction substantially perpendicular to a substantially radial direction relative to the annular body as indicated by arrow B.

To impart sufficient stiffness to the catheter body 84 it is desirable that the device 1 according to the invention comprises an elongated element which imparts sufficient rigidity to allow the device 1 to be pushed into place. In the embodiment two splittable or frangible tubes are provided to impart the desired rigidity. An inner splittable tube 90 is provided on the inner annular wall 3. In the embodiment it is bonded thereto but it will be appreciated any suitable means of fixing may be employed. The tube 90 comprises a series of lengths 91 connected by a series of frangible joints 92. The frangible joints 92 fail under inflation pressure allowing the catheter body 84 to expand to the expanded position (position (B)) with each of the lengths or strips 91 still attached to the inner annular wall 3.

An outer splittable tube 95 is provided on the outer annular wall 4. In the embodiment it is bonded thereto but it will be appreciated any suitable means of fixing may be employed. The tube 95 comprises a series of lengths 96 connected by a series of frangible joints 97. The frangible joints 95 fail under inflation pressure allowing the catheter body 84 to expand to the expanded position (position (B)) with each of the lengths or strips 96 still attached to the outer annular wall 4. The inner and outer splittable tubes 95/96 do not interfere with the collapsing action of the device, and retraction of the device is possible because the device only requires greater rigidity for the insertion (pushing) action.

In use the device of the invention is advanced to the desired site by extending tip 81 and advancing the catheter device 1 in its contracted configuration (FIG. 23 configuration). When the catheter has been advanced to the desired delivery site then the tip 81 is retracted and the catheter is expanded by inflating the non-compliant or semi-compliant balloons 36 (e.g. FIG. 25/26 configuration). The device now has a large central lumen 6 through which any desired medical equipment may be advanced. For example heart valves and the like may be introduced in this way. The catheter opens up a vessel to a desired extent while it can be advanced in a deflated low profile state. By expanding the inflated profile has a dilation effect which allows such introduction. The low profile of the unexpanded configuration allows ease of insertion and substantially reduces the risk of causing any trauma whilst being inserted.

It will be understood by those skilled in the art that any suitable compliant semi-compliant or non-compliant material may be chosen.

For example the non-compliant material, such as is suitable for forming a non-compliant balloon, is selected from (medical-grade) materials well known in the industry, for example, polyethylene, high-density polyethylene, polyethylene terephthalate, polyethylene block amide, polymethylacrylate, polypropylene, polytetrafluoroethylene, expanded polytetrafluoroethylene (ePTFE), polyimide, nylon (polyamide), polyacrylamide, polycarbonate, polyformaldehyde, polyvinylchloride, polyurethane, and the like, and combinations thereof.

Semi-compliant balloons can be fabricated from PET, Nylon, Polyurethane, other thermoplastic elastomers.

For example for compliance (medical-grade) compliant materials are also selected from those well known in the industry, for example, latex (natural rubber), silicone, polyurethane and combinations thereof.

It will be further understood by those skilled in the art that the inflatable device 1 can be implanted permanently or temporarily, depending on the modus operandi of the device, that is, whether the device is required to be in situ for a prolonged period of time or a short period of time. Further, the inflatable device 1 can also include bioresorbable materials such as poly(amino acids), poly(anhydrides), poly(caprolactones), poly(lactic/glycolic acid) polymers, poly(hydroxybutyrates) and poly(orthoesters).

In a further embodiment of the present invention, the inflatable device 1 can also comprise a radiopaque material to allow the device 1 to be tracked when deployed during an radiological intervention, and the like. Alternatively chemical sensors or physical detectors may form part of the device. For example fluoroscopy or ultrasound may be employed.

A device of the invention may be coated or impregnated with a therapeutic material such as an anticoagulant or the like. Where a filter attachment is present it may be desirable for the filter attachment to be additionally or alternatively coated or impregnated with a therapeutic material such as an anticoagulant or the like. Debris caught by the device can be removed by suction or physically broken-up.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. An inflatable device for use within the vasculature of a body and having an expandable annular body which is expandable by inflation of a series of inflation lumen defined therein, the device having inflated and non-inflated states and inner and outer annular walls, the device being adapted so that inflationary pressure within the lumen moves the device from the non-inflated state toward the inflated state by radially outward expansion of both the outer and inner walls so that the annular body expands to form an annular structure with a central lumen defined by the inner wall, and balloons which occupy the lumens.

2. An inflatable device according to claim. 1., wherein the inflation lumen comprise non-compliant or semi-compliant balloons.

3. A device according to claim 1, wherein the annular body is constructed of a compliant material.

4. A device according to claim 1, wherein the annular body is provided by an annular sheath of elastic material with a series of lumen defined therein.

5. (canceled)

6. A device according to claim 1, wherein a plurality of non-compliant or semi-compliant balloons are provided in at least one inflation lumen.

7. (canceled)

8. A device according to claim 1, wherein inflation of the inflatable device is achieved by inflating the balloons.

9. (canceled)

10. (canceled)

11. (canceled)

12. (canceled)

13. A device according to claim 1 wherein the expandable annular body is an annular double-walled balloon comprising:
(i) inner and outer annular balloon walls spaced apart from each other;
(ii) a series of inflation lumen defined between said inner and outer walls.

14. A device according to claim 1, wherein said inflation lumen are defined by partition walls joining the inner and outer walls and wherein the partition walls are constructed of a material that is more resilient to stretching than a material from which the internal or external wall is constructed.

15. (canceled)

16. (canceled)

17. (canceled)

18. (canceled)

19. A device according to claim 1, wherein said inflation lumens are defined by a surface having folds therein.

20. (canceled)

21. A device according to claim 1, wherein a plurality of non-compliant or semi-compliant balloons are in fluid communication with each other so that they may be inflated together.

22. A device according to claim 21, wherein a plurality of non-compliant or semi-compliant balloons are provided in at least one lumen and said non-compliant or semi-compliant balloons are in fluid communication with each other so that they may be inflated together.

23. A device according to claim 21, wherein a plurality of non-compliant or semi-compliant balloons are provided in each of a plurality of lumen and said non-compliant balloons or semi-compliant in a first lumen are in fluid communication with said non-compliant balloons or semi-compliant in a second lumen so that they may be inflated together.

24. (canceled)

25. A device according to claim 1, further comprising at least one non-inflation lumen defined within the body.

26. A device according to claim 1, wherein in the inflated state the central lumen has a diameter of at least about 1 mm.

27. A device according to claim 1, wherein in the non-inflated state the body is stretch-fitted over a delivery device.

28. (canceled)

29. A device according to claim 1, wherein the annular body is tethered proximally to a guide wire.

30. (canceled)

31. A device according to claim 1, comprising a mechanism to stretch the annular body in a substantially axial direction.

32. A device according to claim 1, further comprising a filter which extends across the internal lumen and which can act as an intraluminal embolic capture device to capture embolic material from blood passing through the internal lumen.

33. (canceled)

34. (canceled)

35. A device according to claim 32, wherein the annular body extends over a substantial portion of the length of the filter.

36. A device according to claim 1, further comprising infusion means for infusing a substance into a vessel.

37. A device according to claim 36, wherein the device is adapted to infuse the substance when a predetermined inflated pressure in the annular body is reached.

38. A device according to claim 36, wherein the device comprises one or more rupturable reservoirs which house the substance to be released and wherein the reservoirs are ruptured in use to release the substance.

39. A device according to claim 36, wherein the device comprises infusion tubes on the annular body.

40. (canceled)

41. A device according to claim 39, wherein the device further comprises a perforated sheath optionally of elastic material enveloping the circumference of the annular body and the infusion tubes.

42. A device according to claim 39, wherein the device further comprises a porous sheath enveloping the circumference of the annular body and the infusion tubes.

43. A device according to claim 41, wherein the sheath is bonded to a proximal and distal circumference of the annular body.

44. A device according to claim 1, wherein the device is in the form of a dilation catheter comprising a catheter body.

45. A device according to claim 44, wherein the catheter body is adapted for the delivery of medical devices to a treatment site.

46. A device according to claim 44, wherein the dilation catheter comprises a splittable body which imparts sufficient rigidity to allow the dilation catheter to be pushed into place and which splits apart with expansion of the annular body.

47. A device according to claim 46, comprising a splittable body on the inner or outer annular wall.

48. (canceled)

49. (canceled)
